(19) United States
(12) Patent Application Publication
(10) Pub. No.: US 2004/0170665 A1
(43) Pub. Date: Sep. 2, 2004

(54) Intravitreal botulinum toxin implant

(75) Inventor: Stephen Donovan, Capistrano Beach, CA (US)

Correspondence Address: STEPHEN DONOVAN, ALLERGAN, INC., 2525 Dupont Drive, T2-7H, Irvine, CA, 92612, US

(73) Assignee: Allergan, Inc.

(21) Appl. No.: 10/752,871

(22) Filed: Jan. 6, 2004

Related U.S. Application Data
(63) Continuation-in-part of application No. 10/445,142, filed on May 23, 2003.
Application No. 10/445,142 is a continuation-in-part of application No. 10/096,501, filed on Mar. 11, 2002, now Pat. No. 6,585,993.
Application No. 10/096,501 is a continuation of application No. 09/923,631, filed on Aug. 7, 2001, now Pat. No. 6,383,509.
Application No. 09/923,631 is a continuation of application No. 09/587,250, filed on Jun. 2, 2000, now Pat. No. 6,306,423.

(51) Int. Cl. A61K 39/08; A61F 2/00

(52) U.S. Cl. 424/427; 424/239.1

(57) ABSTRACT

A biodegradable botulinum toxin ocular implant for treating a medical condition of the eye upon implantation of the implant into the vitreous chamber of a patient's eye.

### CROSS REFERENCE

This application is a continuation in part of Ser. No. 10/445,142, filed May 23, 2003, which is a continuation in part of Ser. No. 10/096,501, filed Mar. 11, 2002, now U.S. Pat. No. 6,585,993, which is a continuation of Ser. No. 09/923,631, filed Aug. 7, 2001, now U.S. Pat. No. 6,383,509B1, which is a continuation of Ser. No. 09/587,250, filed Jun. 2, 2000, now U.S. Pat. No. 6,306,423B1. All these applications and patents are incorporated by reference herein in their entireties.

### BACKGROUND

The present invention relates to an ocular drug implant. In particular, the present invention relates to a botulinum toxin ocular implant.

Ocular Disorders

Ocular disorders include macular edema, uveitis, macular degeneration, retinal detachment, ocular tumors, fungal or viral infections, multifocal choroiditis, diabetic retinopathy, proliferative vitreoretinopathy (PVR), sympathetic opthalmia, Vogt Koyanagi-Harada (VKH) syndrome, histoplasmosis, uveal diffusion, vascular occlusion, and the like.

Macular edema (ME) is a nonspecific response of the retina to a variety of insults, and a condition associated with a number of diseases, including uveitis, retinal vascular abnormalities (diabetic retinopathy and retinal venous occlusive disease), a sequela of cataract surgery (Irvine-Gass Syndrome), macular membranes, and inherited or acquired retinal degeneration. Macular edema involves the development of microangiopathy, characterized by abnormal retinal vessel permeability and capillary leakage into the adjacent retinal tissues. The macula becomes thickened due to fluid accumulation from the breakdown of the inner blood-retinal barrier at the level of the capillary endothelium, often resulting in significant disturbances in visual acuity. Blurry vision and decreases in central vision are common.

In many cases macular edema resolves spontaneously or with short-term treatment. However, in cases of persistent macular edema (PME), visual loss continues to be a significant therapeutic challenge. Therapies for macular edema utilize a stepwise approach including surgical and medical methods. Currently there are no approved therapies for the treatment of PME. Macular edema that has failed to respond to drug therapy and laser photocoagulation represents a significant unmet medical need.

Drug therapy includes topical, periocular, subconjunctival/intravitreal, or systemic corticosteroids; topical and systemic nonsteroidal anti-inflammatory botulinum toxins (NSAIDs), and/or immunosuppressants. Nonetheless, with variable incidence, macular edema may persist regardless of treatment or causation resulting in severe vision loss. Retinal toxicity and crystalline retinal deposits following intravitreal triamcinolone acetonide have been reported, suggesting that adequate characterization of potential toxicity and safety are lacking.

Surgical methods for the treatment of macular edema include laser photocoagulation which is administered not withstanding varying results. Focal/grid laser photocoagulation for the prevention of moderate visual loss has been shown to be efficacious in diabetic retinopathy and branch retinal vein occlusion patients, but not in central retinal vein occlusion patients. As a last resort, a vitrectomy is sometimes performed in patients who have persistent macular edema that has failed to respond to less invasive treatments.

Drug Implants

A drug implant can deliver a pharmaceutical in vivo at a predetermined rate over a specific time period. Generally, the release rate of a drug from an implant is a function of the physiochemical properties of the implant material and incorporated drug. Typically, an implant is made of an inert material which elicits little or no host response.

An implant can comprise a drug with a biological activity incorporated into a carrier material. The carrier can be a polymer or a bioceramic material. The implant can be injected, inserted or implanted into a selected location of a patient's body and reside therein for a prolonged period during which the drug is released by the implant in a manner and amount which can impart a desired therapeutic efficacy.

Polymeric carrier materials can release drugs due to diffusion, chemical reaction or solvent activation, as well as upon influence by magnetic, ultrasound or temperature change factors. Diffusion can be from a reservoir or matrix. Chemical control can be due to polymer degradation or cleavage of the drug from the polymer. Solvent activation can involve swelling of the polymer or an osmotic effect. See e.g. Science 249; 1527-1533:1990.

A membrane or reservoir implant depends upon the diffusion of a botulinum toxin across a polymer membrane. A matrix implant is comprised of a polymeric matrix in which the botulinum toxin is uniformly distributed. Swelling-controlled release systems are usually based on hydrophilic, glassy polymers which undergo swelling in the presence of biological fluids or in the presence of certain environmental stimuli.

The implant material used is preferably substantially non-toxic, non-carcinogenic, and non-immunogenic. Suitable implant materials can include polymers such as poly(2-hydroxy ethyl methacrylate) (p-HEMA), poly(N-vinyl pyrrolidone) (p-NVP)+, poly(vinyl alcohol) (PVA), poly(acrylic acid) (PAA), polydimethyl siloxanes (PDMS), ethylene-vinyl acetate copolymers (EVAc), polyvinylpyrrolidone/methylacrylate copolymers, poly(lactic acid) (PLA), poly(glycolic acid) (PGA), polyanhydrides, poly(ortho esters), collagen and cellulosic derivatives and bioceramics, such as hydroxyapatite (HPA), tricalcium phosphate (TCP), and aliminocalcium phosphate (ALCAP). Lactic acid, glycolic acid, collagen and copolymers thereof can be used to make biodegradable implants.

An implant made of a nonbiodegradable polymer has the drawback of requiring both surgical implantation and removal. Hence, biodegradable implants have been used to overcome the evident deficiencies of nonbiodegradable implants. See, e.g., U.S. Pat. Nos. 3,773,919 and 4,767,628. A biodegradable polymer can be a surface eroding polymer, as opposed to a polymer which displays bulk or homogenous degradation. A surface eroding polymer degrades only from its exterior surface, and drug release is therefore proportional to the polymer erosion rate. A suitable such polymer can be a polyanhydride. An implant can be in the form of solid cylindrical implants, pellet microcapsules, or microspheres. Since a biodegradable implant releases drug while degrading there is typically no need to remove the implant. See e.g. Drug Development and Industrial Pharmacy 24(12);1129-1138:1998. A biodegradable implant can be based upon either a membrane or matrix release of the bioactive substance. Biodegradable microspheres can be implanted by injection through a conventional fine needle or pressed into a disc and implanted as a pellet.

Commercially available PLGA (biodegradable) drug incorporating microspheres include the Lupron Depot® (leuprolide acetate), Enantone Depot®, Decapeptil® and Pariodel LA®. Problems with existing microsphere formulations include low encapsulation efficiency, peptide inactivation during the encapsulation process and difficulties in controlling the release kinetics.

A least three methods for preparing polymeric microspheres, including microspheres composed of a biodegradable polymer, are known. See e.g. Journal of Controlled Release 52(3);227-237:1998. Thus, a solid drug preparation can be dispersed into a continuous phase consisting of a biodegradable polymer in an organic solvent or, an aqueous solution of a drug can be emulsified into the polymer-organic phase. Microspheres can then be formed by spray-drying, phase separation or double emulsion techniques.

Hydrogels have been used to construct single pulse and multiple pulse drug delivery implants. A single pulse implant can be osmotically controlled or melting controlled. Doelker E., Cellulose Derivatives, Adv Polym Sci 107; 199-265:1993. It is known that multiple pulses of certain substances from an implant can be achieved in response to an environmental change in a parameter such as temperature (Mater Res Soc Symp Proc, 331;211-216:1994; J. Contr Rel 15;141-152:1991), pH (Mater Res Soc Symp Proc, 331;199-204:1994), ionic strength (React Polym, 25;127-137:1995), magnetic fields (J. Biomed Mater Res, 21; 1367-1373:1987) or ultrasound.

Anti-inflammatory drugs are routinely administered by topical or oral routes for the treatment of uveitis of various etiologies. However, topical and/or oral drug administration often fails to achieve an adequate intraocular drug concentration. Thus, poor intraocular penetration of topical medications into the posterior segment of the eye is a well known problem. The high drug plasma levels required to achieve an adequate intraocular drug level often causes systemic side effects such as hypertension, hyperglycemia, increased susceptibility to infection, peptic ulcers, psychosis, and other complications.

The most efficient means of delivering a drug to the posterior segment is by direct delivery of the drug into the vitreous chamber. By delivering a drug intravitreally, the blood-eye barrier is circumvented and intraocular therapeutic levels can be achieved without the risk of systemic toxicity. Unfortunately though, the natural pharmacokinetics of the eye typically result in a short half-life unless the drug can be delivered using a formulation capable of providing a sustained release of the drug.

Sustained release intravitreal drug containing implants are known. Thus, for example, the controlled release of drugs from polylactide/polyglycolide (PLGA) copolymers into the vitreous is known. See e.g. U.S. Pat. No. 5,501,856 and EP 654,256. Additionally, intravitreal dexamethasone implants have been used or proposed for use to treat various ocular conditions. See e.g. U.S. patent application Ser. Nos. 09/693,008; 09/997,094, and; 10/327,018. Dexamethasone is known to possess potent anti-inflammatory activity.

Protein Implants

Implants for the release of various macromolecules are known. Thus, biocompatible, polymeric pellets which incorporate a high molecular weight protein have been implanted and shown to exhibit continuous release of the protein for periods exceeding 100 days. Additionally, various labile, high molecular weight enzymes (such as alkaline phosphatase, molecular weight 88 kD and catalase, molecular weight 250 kD) have been incorporated into biocompatible, polymeric implants with long term, continuous release characteristics. Generally an increase in the polymer concentration in the casting solution decreases the initial rate at which protein is released from the implant. Nature 263; 797-800:1976.

Furthermore, it is known that albumin can be released from an EVAc implant and polylysine can be released from collagen based microspheres. Mallapragada S. K. et al, at page 431 of chapter 27 in Von Recum, A. F. Handbook of Biomaterials Evaluation, second edition, Taylor & Francis (1999). Additionally, the release of tetanus toxoid from microspheres has been studied. Ibid at 432. Sintered EVAc copolymer inserted subcutaneously has been shown to release insulin over a period of 100 days. Ibid at 433.

Proteins, such as human growth hormone (hGH) (molecular weight about 26 kD), have been encapsulated within a polymeric matrix which when implanted permits the human growth hormone to be released in vivo over a period of about a week. See e.g. U.S. Pat. No. 5,667,808.

The concept of controlled release antigen delivery systems has been the subject of intensive research efforts. A motivation for this work has been the development of continuous and pulsatile release vaccine delivery systems whereby long lasting protection through immunization can be provided through a single dose system as opposed to multiple, separate dosing vaccine administration schedules. Thus, vaccine delivery systems which can provide effective immunization after a single administration of the antigen delivery system have been sought. Many studies on vaccine delivery systems have been carried out with bacterial toxins, such as tetanus toxoid. See infra.

A protein incorporating implant can exhibit an initial burst of protein release, followed by a generally monophasic release thereafter. Unfortunately, due to the high concentration of protein within a controlled release matrix, the protein molecules can exhibit a tendency to aggregate and form denatured, immunogenic concentrations of protein.

Biodegradable microspheres implants for pulsatile release of a protein toxoid, such as a vaccine, are known. Thus, a solvent evaporation process has been used to make biodegradable, poly(lactic-co-glycolic acid) (PLGA) microspheres capable of providing either a continuous delivery of therapeutic proteins or a pulsatile delivery of protein vaccines with a triphasic release pattern. Biotechnol Prog 14(1):102-7:1998.

Additionally, biodegradable PLGA microspheres capable of pulsatile release of protein antigens, wherein the first pulse or pulse and the second pulse of antigen can be spaced by up to about six months apart are known. Hanes, J. et al., New Advances in Microsphere-Based Single-Dose Vaccines, Adv Drug Del Rev 28;97-119:1997.

Significantly, pulsed administration of a subunit vaccine (a recombinant glycoprotein) to HIV has been accomplished using poly(lactic-co-glycolic) acid (PLGA) microspheres. The immunizing pulses of protein vaccine can be timed to take place up to six month after implantation, such subsequent pulses of an antigen eliminating the need for repeated immunizations. J Pharm Sci 87(12):1489-95:1998.

Botulinum Toxin

The genus Clostridium has more than one hundred and twenty seven species, grouped according to their morphology and functions. The anaerobic, gram positive bacterium Clostridium botulinum produces a potent polypeptide neurotoxin, botulinum toxin, which causes a neuroparalytic illness in humans and animals referred to as botulism. The spores of Clostridium botulinum are found in soil and can grow in improperly sterilized and sealed food containers of home based canneries, which are the cause of many of the cases of botulism. The effects of botulism typically appear 18 to 36 hours after eating the foodstuffs infected with a Clostridium botulinum culture or spores. The botulinum toxin can apparently pass unattenuated through the lining of the gut and attack peripheral motor neurons. Symptoms of botulinum toxin intoxication can progress from difficulty walking, swallowing, and speaking to paralysis of the respiratory muscles and death.

Botulinum toxin type A is the most lethal natural biological agent known to man. About 50 picograms of a commercially available botulinum toxin type A (purified neurotoxin complex)¹ is a LD₅₀ in mice (i.e. 1 unit). One unit of BOTOX® contains about 50 picograms (about 56 attomoles) of botulinum toxin type A complex. Interestingly, on a molar basis, botulinum toxin type A is about 1.8 billion times more lethal than diphtheria, about 600 million times more lethal than sodium cyanide, about 30 million times more lethal than cobra toxin and about 12 million times more lethal than cholera. Singh, Critical Aspects of Bacterial Protein Toxins, pages 63-84 (chapter 4) of Natural Toxins II, edited by B. R. Singh et al., Plenum Press, New York (1976) (where the stated LD₅₀ of botulinum toxin type A of 0.3 ng equals 1 U is corrected for the fact that about 0.05 ng of BOTOX® equals 1 unit). One unit (U) of botulinum toxin is defined as the LD₅₀ upon intraperitoneal injection into female Swiss Webster mice weighing 18 to 20 grams each. ¹ Available from Allergan, Inc., of Irvine, Calif. under the tradename BOTOX® in 100 unit vials)

Seven generally immunologically distinct botulinum neurotoxins have been characterized, these being respectively botulinum neurotoxin serotypes A, B, C₁, D, E, F and G each of which is distinguished by neutralization with type-specific antibodies. The different serotypes of botulinum toxin vary in the animal species that they affect and in the severity and duration of the paralysis they evoke. For example, it has been determined that botulinum toxin type A is 500 times more potent, as measured by the rate of paralysis produced in the rat, than is botulinum toxin type B. Additionally, botulinum toxin type B has been determined to be non-toxic in primates at a dose of 480 U/kg which is about 12 times the primate LD₅₀ for botulinum toxin type A. Moyer E et al., Botulinum Toxin Type B: Experimental and Clinical Experience, being chapter 6, pages 71-85 of “Therapy With Botulinum Toxin”, edited by Jankovic, J. et al. (1994), Marcel Dekker, Inc. Botulinum toxin apparently binds with high affinity to cholinergic motor neurons, is translocated into the neuron and blocks the release of acetylcholine. Additional uptake can take place through low affinity receptors, as well as by phagocytosis and pinocytosis.

Regardless of serotype, the molecular mechanism of toxin intoxication appears to be similar and to involve at least three steps or stages. In the first step of the process, the toxin binds to the presynaptic membrane of the target neuron through a specific interaction between the heavy chain, H chain, and a cell surface receptor; the receptor is thought to be different for each type of botulinum toxin and for tetanus toxin. The carboxyl end segment of the H chain, H_{C}, appears to be important for targeting of the toxin to the cell surface.

In the second step, the toxin crosses the plasma membrane of the poisoned cell. The toxin is first engulfed by the cell through receptor-mediated endocytosis, and an endosome containing the toxin is formed. The toxin then escapes the endosome into the cytoplasm of the cell. This step is thought to be mediated by the amino end segment of the H chain, H_{N}, which triggers a conformational change of the toxin in response to a pH of about 5.5 or lower. Endosomes are known to possess a proton pump which decreases intra-endosomal pH. The conformational shift exposes hydrophobic residues in the toxin, which permits the toxin to embed itself in the endosomal membrane. The toxin (or at a minimum the light chain) then translocates through the endosomal membrane into the cytoplasm.

The last step of the mechanism of botulinum toxin activity appears to involve reduction of the disulfide bond joining the heavy chain, H chain, and the light chain, L chain. The entire toxic activity of botulinum and tetanus toxins is contained in the L chain of the holotoxin; the L chain is a zinc (Zn++) endopeptidase which selectively cleaves proteins essential for recognition and docking of neurotransmitter-containing vesicles with the cytoplasmic surface of the plasma membrane, and fusion of the vesicles with the plasma membrane. Tetanus neurotoxin, botulinum toxin types B, D, F, and G cause degradation of synaptobrevin (also called vesicle-associated membrane protein (VAMP)), a synaptosomal membrane protein. Most of the VAMP present at the cytoplasmic surface of the synaptic vesicle is removed as a result of any one of these cleavage events. Botulinum toxin serotype A and E cleave SNAP-25. Botulinum toxin serotype C₁ was originally thought to cleave syntaxin, but was found to cleave syntaxin and SNAP-25. Each of the botulinum toxins specifically cleaves a different bond, except botulinum toxin type B (and tetanus toxin) which cleave the same bond. Each of these cleavages block the process of vesicle-membrane docking, thereby preventing exocytosis of vesicle content.

Although all the botulinum toxins serotypes apparently inhibit release of the neurotransmitter acetylcholine at the neuromuscular junction, they do so by affecting different neurosecretory proteins and/or cleaving these proteins at different sites. For example, botulinum types A and E both cleave the 25 kiloDalton (kD) synaptosomal associated protein (SNAP-25), but they target different amino acid sequences within this protein. Botulinum toxin types B, D, F and G act on vesicle-associated protein (VAMP, also called synaptobrevin), with each serotype cleaving the protein at a different site. Finally, botulinum toxin type C₁ has been shown to cleave both syntaxin and SNAP-25. These differences in mechanism of action may affect the relative potency and/or duration of action of the various botulinum toxin serotypes. Apparently, a substrate for a botulinum toxin can be found in a variety of different cell types. See e.g. Biochem J 1;339 (pt 1):159-65:1999, and Mov Disord, 10(3):376:1995 (pancreatic islet B cells contains at least SNAP-25 and synaptobrevin).

The molecular weight of the botulinum toxin protein molecule, for all seven of the known botulinum toxin serotypes, is about 150 kD. Interestingly, the botulinum toxins are released by Clostridial bacterium as complexes comprising the 150 kD botulinum toxin protein molecule along with associated non-toxin proteins. Thus, the botulinum toxin type A complex can be produced by Clostridial bacterium as 900 kD, 500 kD and 300 kD forms. Botulinum toxin types B and C₁ is apparently produced as only a 700 kD or 500 kD complex. Botulinum toxin type D is produced as both 300 kD and 500 kD complexes. Finally, botulinum toxin types E and F are produced as only approximately 300 kD complexes. The complexes (i.e. molecular weight greater than about 150 kD) are believed to contain a non-toxin hemaglutinin protein and a non-toxin and non-toxic nonhemaglutinin protein. These two non-toxin proteins (which along with the botulinum toxin molecule comprise the relevant neurotoxin complex) may act to provide stability against denaturation to the botulinum toxin molecule and protection against digestive acids when toxin is ingested. Additionally, it is possible that the larger (greater than about 150 kD molecular weight) botulinum toxin complexes may result in a slower rate of diffusion of the botulinum toxin away from a site of intramuscular injection of a botulinum toxin complex.

In vitro studies have indicated that botulinum toxin inhibits potassium cation induced release of both acetylcholine and norepinephrine from primary cell cultures of brainstem tissue. Additionally, it has been reported that botulinum toxin inhibits the evoked release of both glycine and glutamate in primary cultures of spinal cord neurons and that in brain synaptosome preparations botulinum toxin inhibits the release of each of the neurotransmitters acetylcholine, dopamine, norepinephrine (Habermann E., et al., Tetanus Toxin and Botulinum A and C Neurotoxins Inhibit Noradrenaline Release From Cultured Mouse Brain, J Neurochem 51(2);522-527:1988) CGRP, substance P and glutamate (Sanchez-Prieto, J., et al., Botulinum Toxin A Blocks Glutamate Exocytosis From Guinea Pig Cerebral Cortical Synaptosomes, Eur J. Biochem 165;675-681:1897. Thus, when adequate concentrations are used, stimulus-evoked release of most neurotransmitters is blocked by botulinum toxin. See e.g. Pearce, L. B., Pharmacologic Characterization of Botulinum Toxin For Basic Science and Medicine, Toxicon 35(9);1373-1412 at 1393; Bigalke H., et al., Botulinum A Neurotoxin Inhibits Non-Cholinergic Synaptic Transmission in Mouse Spinal Cord Neurons in Culture, Brain Research 360;318-324:1985; Habermann E., Inhibition by Tetanus and Botulinum A Toxin of the release of [³H]Noradrenaline and [³H]GABA From Rat Brain Homogenate, Experientia 44;224-226:1988, Bigalke H., et al., Tetanus Toxin and Botulinum A Toxin Inhibit Release and Uptake of Various Transmitters, as Studied with Particulate Preparations From Rat Brain and Spinal Cord, Naunyn-Schmiedeberg's Arch Pharmacol 316;244-251:1981, and; Jankovic J. et al., Therapy With Botulinum Toxin, Marcel Dekker, Inc., (1994), page 5.

Botulinum toxin type A can be obtained by establishing and growing cultures of Clostridium botulinum in a fermenter and then harvesting and purifying the fermented mixture in accordance with known procedures. All the botulinum toxin serotypes are initially synthesized as inactive single chain proteins which must be cleaved or nicked by proteases to become neuroactive. The bacterial strains that make botulinum toxin serotypes A and G possess endogenous proteases and serotypes A and G can therefore be recovered from bacterial cultures in predominantly their active form. In contrast, botulinum toxin serotypes C₁, D and E are synthesized by nonproteolytic strains and are therefore typically unactivated when recovered from culture. Serotypes B and F are produced by both proteolytic and nonproteolytic strains and therefore can be recovered in either the active or inactive form. However, even the proteolytic strains that produce, for example, the botulinum toxin type B serotype only cleave a portion of the toxin produced. The exact proportion of nicked to unnicked molecules depends on the length of incubation and the temperature of the culture. Therefore, a certain percentage of any preparation of, for example, the botulinum toxin type B toxin is likely to be inactive, possibly accounting for the known significantly lower potency of botulinum toxin type B as compared to botulinum toxin type A. The presence of inactive botulinum toxin molecules in a clinical preparation will contribute to the overall protein load of the preparation, which has been linked to increased antigenicity, without contributing to its clinical efficacy. Additionally, it is known that botulinum toxin type B has, upon intramuscular injection, a shorter duration of activity and is also less potent than botulinum toxin type A at the same dose level.

High quality crystalline botulinum toxin type A can be produced from the Hall A strain of Clostridium botulinum with characteristics of >3×10⁷ U/mg, an A₂₆₀/A₂₇₈ of less than 0.60 and a distinct pattern of banding on gel electrophoresis. The known Shantz process can be used to obtain crystalline botulinum toxin type A, as set forth in Shantz, E. J., et al, Properties and use of Botulinum toxin and Other Microbial Neurotoxins in Medicine, Microbiol Rev. 56;80-99:1992. Generally, the botulinum toxin type A complex can be isolated and purified from an anaerobic fermentation by cultivating Clostridium botulinum type A in a suitable medium. The known process can also be used, upon separation out of the non-toxin proteins, to obtain pure botulinum toxins, such as for example: purified botulinum toxin type A with an approximately 150 kD molecular weight with a specific potency of 1-2×10⁸ LD₅₀ U/mg or greater; purified botulinum toxin type B with an approximately 156 kD molecular weight with a specific potency of 1-2×10⁸ LD₅₀ U/mg or greater, and; purified botulinum toxin type F with an approximately 155 kD molecular weight with a specific potency of 1-2×10⁷ LD₅₀ U/mg or greater.

Botulinum toxins and/or botulinum toxin complexes can be obtained from List Biological Laboratories, Inc., Campbell, Calif.; the Centre for Applied Microbiology and Research, Porton Down, U.K.; Wako (Osaka, Japan), Metabiologics (Madison, Wis.) as well as from Sigma Chemicals of St Louis, Mo. Pure botulinum toxin can also be used to prepare a pharmaceutical composition.

As with enzymes generally, the biological activities of the botulinum toxins (which are intracellular peptidases) is dependant, at least in part, upon their three dimensional conformation. Thus, botulinum toxin type A is detoxified by heat, various chemicals surface stretching and surface drying. Additionally, it is known that dilution of the toxin complex obtained by the known culturing, fermentation and purification to the much, much lower toxin concentrations used for pharmaceutical composition formulation results in rapid detoxification of the toxin unless a suitable stabilizing agent is present. Dilution of the toxin from milligram quantities to a solution containing nanograms per milliliter presents significant difficulties because of the rapid loss of specific toxicity upon such great dilution. Since the toxin may be used months or years after the toxin containing pharmaceutical composition is formulated, the toxin can stabilized with a stabilizing agent such as albumin and gelatin.

The success of botulinum toxin type A to treat a variety of clinical conditions has led to interest in other botulinum toxin serotypes. Two commercially available botulinum type A preparations for use in humans are BOTOX® available from Allergan, Inc., of Irvine, Calif., and Dysport® available from Beaufour Ipsen, Porton Down, England. A Botulinum toxin type B preparation (MyoBloc®) is available from Elan Pharmaceuticals of San Francisco, Calif.

BOTOX® consists of a purified botulinum toxin type A complex, albumin and sodium chloride packaged in sterile, vacuum-dried form. The botulinum toxin type A is made from a culture of the Hall strain of Clostridium botulinum grown in a medium containing N-Z amine and yeast extract. The botulinum toxin type A complex is purified from the culture solution by a series of acid precipitations to a crystalline complex consisting of the active high molecular weight toxin protein and an associated hemagglutinin protein. The crystalline complex is re-dissolved in a solution containing saline and albumin and sterile filtered (0.2 microns) prior to vacuum-drying. The vacuum-dried product is stored in a freezer at or below −5° C. BOTOX® can be reconstituted with sterile, non-preserved saline prior to intramuscular injection. Each vial of BOTOX® contains about 100 units (U) of Clostridium botulinum toxin type A purified neurotoxin complex, 0.5 milligrams of human serum albumin and 0.9 milligrams of sodium chloride in a sterile, vacuum-dried form without a preservative.

To reconstitute vacuum-dried BOTOX®, sterile normal saline without a preservative; (0.9% Sodium Chloride Injection) is used by drawing up the proper amount of diluent in the appropriate size syringe. Since BOTOX® may be denatured by bubbling or similar violent agitation, the diluent is gently injected into the vial. For sterility reasons BOTOX® is preferably administered within four hours after the vial is removed from the freezer and reconstituted. During these four hours, reconstituted BOTOX® can be stored in a refrigerator at about 2° C. to about 8° C. Reconstituted, refrigerated BOTOX® has been reported to retain its potency for at least about two weeks. Neurology, 48:249-53:1997.

Botulinum toxins have been used in clinical settings for the treatment of neuromuscular disorders characterized by hyperactive skeletal muscles (i.e. motor disorders). In 1989 a botulinum toxin type A complex has been approved by the U.S. Food and Drug Administration for the treatment of blepharospasm, strabismus and hemifacial spasm. Subsequently, a botulinum toxin type A was also approved by the FDA for the treatment of cervical dystonia and for the treatment of glabellar lines, and a botulinum toxin type B was approved for the treatment of cervical dystonia. Non-type A botulinum toxin serotypes apparently have a lower potency and/or a shorter duration of activity as compared to botulinum toxin type A. Clinical effects of peripheral intramuscular botulinum toxin type A are usually seen within one week of injection. The typical duration of symptomatic relief from a single intramuscular injection of botulinum toxin type A averages about three months, although significantly longer periods of therapeutic activity have been reported.

It has been reported that botulinum toxin type A has been used in clinical settings as follows:

(1) about 75-125 units of BOTOX® per intramuscular injection (multiple muscles) to treat cervical dystonia;

(2) 5-10 units of BOTOX® per intramuscular injection to treat glabellar lines (brow furrows) (5 units injected intramuscularly into the procerus muscle and 10 units injected intramuscularly into each corrugator supercilii muscle);

(3) about 30-80 units of BOTOX® to treat constipation by intrasphincter injection of the puborectalis muscle;

(4) about 1-5 units per muscle of intramuscularly injected BOTOX® to treat blepharospasm by injecting the lateral pre-tarsal orbicularis oculi muscle of the upper lid and the lateral pre-tarsal orbicularis oculi of the lower lid.

(5) to treat strabismus, extraocular muscles have been injected intramuscularly with between about 1-5 units of BOTOX®, the amount injected varying based upon both the size of the muscle to be injected and the extent of muscle paralysis desired (i.e. amount of diopter correction desired).

(6) to treat upper limb spasticity following stroke by intramuscular injections of BOTOX® into five different upper limb flexor muscles, as follows:

(a) flexor digitorum profundus: 7.5 U to 30 U

(b) flexor digitorum sublimus: 7.5 U to 30 U

(c) flexor carpi ulnaris: 10 U to 40 U

(d) flexor carpi radialis: 15 U to 60 U

(e) biceps brachii: 50 U to 200 U. Each of the five indicated muscles has been injected at the same treatment session, so that the patient receives from 90 U to 360 U of upper limb flexor muscle BOTOX® by intramuscular injection at each treatment session.

(7) to treat migraine, pericranial injected (injected symmetrically into glabellar, frontalis and temporalis muscles) injection of 25 U of BOTOX® has showed significant benefit as a prophylactic treatment of migraine compared to vehicle as measured by decreased measures of migraine frequency, maximal severity, associated vomiting and acute medication use over the three month period following the 25 U injection.

(8) to treat neuropathic pain syndromes such as complex regional pain syndrome (CRPS) by injecting 300 U of BOTOX® into the sternocleidomastoid, trapezius, splenius capitis, splenius cervicis, levator scapular, supraspinatus, infraspinatus, or rhomboid major muscle groups. See e.g. Argoff, A Focused Review on the Use of Botulinum Toxins for Neuropathic Pain, Clin J Pain 18(6 Suppl);S177-S181:2002.

(9) to treat cervical spinal cord injuries with multiple subcutaneous injections (about 16-20) of 5 U (a total dose approximately of 100 U) of BOTOX®. Ibid.

(10) to treat postherpetic neuralgia (PHN) using 5 U of BOTOX® per 0.1 ml of normal saline for every 9 cm of painful skin (total doses did not exceed 200 U). Ibid.

It is known that botulinum toxin type A can have an efficacy for up to 12 months (European J Neurology 6 (Supp 4): S111-S1150:1999), and in some circumstances for as long as 27 months, when used to treat glands, such as in the treatment of hyperhydrosis. See e.g. Bushara K., Botulinum toxin and rhinorrhea, Otolaryngol Head Neck Surg 1996;114(3):507, and The Laryngoscope 109:1344-1346:1999. However, the usual duration of an intramuscular injection of Botox® is typically about 3 to 4 months.

In addition to having pharmacologic actions at the peripheral location, botulinum toxins may also have inhibitory effects in the central nervous system. Work by Weigand et al, Nauny-Schmiedeberg's Arch. Pharmacol. 1976; 292, 161-165, and Habermann, Nauny-Schmiedeberg's Arch. Pharmacol. 1974; 281, 47-56 showed that botulinum toxin is able to ascend to the spinal area by retrograde transport. As such, a botulinum toxin injected at a peripheral location, for example intramuscularly, may be retrograde transported to the spinal cord.

A botulinum toxin has also been proposed for the treatment of rhinorrhea (chronic discharge from the nasal mucous membranes, i.e. runny nose), rhinitis (inflammation of the nasal mucous membranes), hyperhydrosis and other disorders mediated by the autonomic nervous system (U.S. Pat. No. 5,766,605), tension headache, (U.S. Pat. No. 6,458,365), migraine headache (U.S. Pat. No. 5,714,468), post-operative pain and visceral pain (U.S. Pat. No. 6,464,986), pain treatment by intraspinal toxin administration (U.S. Pat. No. 6,113,915), Parkinson's disease and other diseases with a motor disorder component, by intracranial toxin administration (U.S. Pat. No. 6,306,403), hair growth and hair retention (U.S. Pat. No. 6,299,893), psoriasis and dermatitis (U.S. Pat. No. 5,670,484), injured muscles (U.S. Pat. No. 6,423,319, various cancers (U.S. Pat. No. 6,139,845), pancreatic disorders (U.S. Pat. No. 6,143,306), smooth muscle disorders (U.S. Pat. No. 5,437,291, including injection of a botulinum toxin into the upper and lower esophageal, pyloric and anal sphincters)), prostate disorders (U.S. Pat. No. 6,365,164), inflammation, arthritis and gout (U.S. Pat. No. 6,063,768), juvenile cerebral palsy (U.S. Pat. No. 6,395,277), inner ear disorders (U.S. Pat. No. 6,265,379), thyroid disorders (U.S. Pat. No. 6,358,513), parathyroid disorders (U.S. Pat. No. 6,328,977) and neurogenic inflammation (U.S. Pat. No. 6,063,768). Additionally, controlled release toxin implants are known (see e.g. U.S. Pat. Nos. 6,306,423 and 6,312,708).

Tetanus toxin, as wells as derivatives (i.e. with a non-native targeting moiety), fragments, hybrids and chimeras thereof can also have therapeutic utility. The tetanus toxin bears many similarities to the botulinum toxins. Thus, both the tetanus toxin and the botulinum toxins are polypeptides made by closely related species of Clostridium (Clostridium tetani and Clostridium botulinum, respectively). Additionally, both the tetanus toxin and the botulinum toxins are dichain proteins composed of a light chain (molecular weight about 50 kD) covalently bound by a single disulfide bond to a heavy chain (molecular weight about 100 kD). Hence, the molecular weight of tetanus toxin and of each of the seven botulinum toxins (non-complexed) is about 150 kD. Furthermore, for both the tetanus toxin and the botulinum toxins, the light chain bears the domain which exhibits intracellular biological (protease) activity, while the heavy chain comprises the receptor binding (immunogenic) and cell membrane translocational domains.

Further, both the tetanus toxin and the botulinum toxins exhibit a high, specific affinity for gangliocide receptors on the surface of presynaptic cholinergic neurons. Receptor mediated endocytosis of tetanus toxin by peripheral cholinergic neurons results in retrograde axonal transport, blocking of the release of inhibitory neurotransmitters from central synapses and a spastic paralysis. Contrarily, receptor mediated endocytosis of botulinum toxin by peripheral cholinergic neurons results in little if any retrograde transport, inhibition of acetylcholine exocytosis from the intoxicated peripheral motor neurons and a flaccid paralysis.

Finally, the tetanus toxin and the botulinum toxins resemble each other in both biosynthesis and molecular architecture. Thus, there is an overall 34% identity between the protein sequences of tetanus toxin and botulinum toxin type A, and a sequence identity as high as 62% for some functional domains. Binz T. et al., The Complete Sequence of Botulinum Neurotoxin Type A and Comparison with Other Clostridial Neurotoxins, J Biological Chemistry 265(16);9153-9158:1990.

Acetylcholine

Typically only a single type of small molecule neurotransmitter is released by each type of neuron in the mammalian nervous system, although there is evidence which suggests that several neuromodulators can be released by the same neuron. The neurotransmitter acetylcholine is secreted by neurons in many areas of the brain, but specifically by the large pyramidal cells of the motor cortex, by several different neurons in the basal ganglia, by the motor neurons that innervate the skeletal muscles, by the preganglionic neurons of the autonomic nervous system (both sympathetic and parasympathetic), by the bag 1 fibers of the muscle spindle fiber, by the postganglionic neurons of the parasympathetic nervous system, and by some of the postganglionic neurons of the sympathetic nervous system. Essentially, only the postganglionic sympathetic nerve fibers to the sweat glands, the piloerector muscles and a few blood vessels are cholinergic as most of the postganglionic neurons of the sympathetic nervous system secret the neurotransmitter norepinephine. In most instances acetylcholine has an excitatory effect. However, acetylcholine is known to have inhibitory effects at some of the peripheral parasympathetic nerve endings, such as inhibition of heart rate by the vagal nerve.

The efferent signals of the autonomic nervous system are transmitted to the body through either the sympathetic nervous system or the parasympathetic nervous system. The preganglionic neurons of the sympathetic nervous system extend from preganglionic sympathetic neuron cell bodies located in the intermediolateral horn of the spinal cord. The preganglionic sympathetic nerve fibers, extending from the cell body, synapse with postganglionic neurons located in either a paravertebral sympathetic ganglion or in a prevertebral ganglion. Since, the preganglionic neurons of both the sympathetic and parasympathetic nervous system are cholinergic, application of acetylcholine to the ganglia will excite both sympathetic and parasympathetic postganglionic neurons.

Acetylcholine activates two types of receptors, muscarinic and nicotinic receptors. The muscarinic receptors are found in all effector cells stimulated by the postganglionic, neurons of the parasympathetic nervous system as well as in those stimulated by the postganglionic cholinergic neurons of the sympathetic nervous system. The nicotinic receptors are found in the adrenal medulla, as well as within the autonomic ganglia, that is on the cell surface of the postganglionic neuron at the synapse between the preganglionic and postganglionic neurons of both the sympathetic and parasympathetic systems. Nicotinic receptors are also found in many nonautonomic nerve endings, for example in the membranes of skeletal muscle fibers at the neuromuscular junction.

Acetylcholine is released from cholinergic neurons when small, clear, intracellular vesicles fuse with the presynaptic neuronal cell membrane. A wide variety of non-neuronal secretory cells, such as, adrenal medulla (as well as the PC12 cell line) and pancreatic islet cells release catecholamines and parathyroid hormone, respectively, from large dense-core vesicles. The PC12 cell line is a clone of rat pheochromocytoma cells extensively used as a tissue culture model for studies of sympathoadrenal development. Botulinum toxin inhibits the release of both types of compounds from both types of cells in vitro, permeabilized (as by electroporation) or by direct injection of the toxin into the denervated cell. Botulinum toxin is also known to block release of the neurotransmitter glutamate from cortical synaptosomes cell cultures.

A neuromuscular junction is formed in skeletal muscle by the proximity of axons to muscle cells. A signal transmitted through the nervous system results in an action potential at the terminal axon, with activation of ion channels and resulting release of the neurotransmitter acetylcholine from intraneuronal synaptic vesicles, for example at the motor endplate of the neuromuscular junction. The acetylcholine crosses the extracellular space to bind with acetylcholine receptor proteins on the surface of the muscle end plate. Once sufficient binding has occurred, an action potential of the muscle cell causes specific membrane ion channel changes, resulting in muscle cell contraction. The acetylcholine is then released from the muscle cells and metabolized by cholinesterases in the extracellular space. The metabolites are recycled back into the terminal axon for reprocessing into further acetylcholine.

Antinociceptive Properties of Botulinum Toxin

Botulinum toxin can affect neurons within the CNS. For example, botulinum toxin serotypes B and F and tetanus toxin are internalized by cultured rat hippocampal astrocytes and cleave the appropriate substrate. Neuropeptide release was reported to be inhibited by botulinum toxin (botulinum toxins A, B, C1, F) treatment in vitro from embryonic rat dorsal root ganglia neurons and from isolated rabbit iris sphincter and dilatory muscles. More importantly, the in vitro release of acetylcholine and substance P (but not norepinephrine) from the rabbit ocular tissue was also inhibited with botulinum toxin A. Therefore, based on these in vitro and limited in vivo data, it can be hypothesized that botulinum toxin treatment may reduce the local release of nociceptive neuropeptides from either cholinergic neurons or from C or A delta fibers in vivo. The reduced neuropeptide release could prevent the local sensitization of nociceptors and thus reduce the perception of pain. A reduction of nociceptive signals from the periphery could then reduce the central sensitization associated with chronic pain. This effect on the nociceptive neurons could work in concert with the other well-known effects of botulinum toxin on the cholinergic motor neuron innervating the extrafusal and intrafusal fibers.

Botulinum toxin therapy has been reported to alleviate pain associated with various conditions with or without concomitant excess muscle contractions. Aoki K. R., Pharmacology and immunology of botulinum toxin serotypes, J Neurol 248(suppl 1);1/3-1/10:2001. Early observations in patients with cervical dystonia who were treated with BOTOX® suggested that the pain relief exceeded the motor benefit. In other areas, the pain associated with myoclonus of spinal cord origin has been treated effectively with BOTOX®. Tension-associated headaches have been reported to be alleviated with BOTOX® therapy. In a double-blind placebo-controlled trial, investigators reported profound antinociceptive activity of intramuscular BOTOX® when administered prior to aductor-release surgery in children with cerebral palsy. The effect was so dramatic that the trial was terminated early. Children treated with BOTOX® had a reduced need for narcotic analgesics, were discharged earlier, and had better outcomes than the placebo group. In a pilot study, patients with chronic whiplash-associated neck pain were successfully treated with BOTOX®. Other reports of BOTOX® for reduction of primary pain include trigger point injections, myofascial pain and migraine headache prophylaxis, and back pain.

A preclinical investigation on the local antinociceptive efficacy of BOTOX® has been reported. A rat model of inflammatory pain was used to demonstrate that a subcutaneous injection of BOTOX® prevented the classical behavioral pain response to a subplantar injection of formalin. Cui M, Aoki K R, Botulinum toxin type a (BTX-a) reduced inflammatory pain in the rat formalin model, Cephalalgia 20(4);414:2000. BOTOX® (3.5 and 7 units/kg) was administered subcutaneously to the plantar surface of the rat 5 days before the formalin challenge in the same area. BOTOX® produced local antinociceptive effects without obvious muscle weakness.

BOTOX® has also been shown to dose dependently inhibit formalin-induced glutamate release in the rat paw and the expression of C-fos in the dorsal horn of the spinal cord. Cui M, Li Z, You S, Khanijou S, Aoki K R, Mechanisms of Antinociceptive Effect of Subcutaneous BOTOX®: Inhibition of Peripheral and Central Nociceptive Processing, Naunyn Schmiedegergs Arch Pharmacol 265(Suppl 2);R17:2002. BOTOX® has also been shown to inhibit calcitonin gene-related peptide (CGRP) release from trigeminal ganglia nerves. Durham P, Cady R, Cady R, Mechanism of botulinum toxin type-A Inhibition of Calcitonin Gene-Related Peptide Secretion from Trigeminal Nerve Cells, Cephalalgia 23(7);690:2003. Using microdialysis, it was found that BOTOX® inhibited capsaicin-induced thermal hyperalgesia suggesting an action on substance P. Aoki K R, Cui M, Mechanisms of the Antinociceptive Effect of Subcutaneous BOTOX®: Inhibition of Peripheral and Central Nociceptive Processing, Cephalalgia 23(7);649:2003. These results indicate that subcutaneous BOTOX® inhibits neurotransmitter release from primary sensory neurons in the rat formalin model. Through this mechanism, BOTOX® inhibits peripheral sensitization in these models, which leads to an indirect reduction in central sensitization.

The preclinical (in vitro and in vivo) evidence coupled with the clinical observations strongly suggests that botulinum toxin (especially BOTOX®) may have a separate antinociceptive effect from its well-known effect on the neuromuscular junction and other cholinergic nerves.

Tetanus Toxoid Implants

The tetanus toxin bears many similarities to the botulinum toxins. Thus, both the tetanus toxin and the botulinum toxins are polypeptides made by closely related species of Clostridium (Clostridium tetani and Clostridium botulinum, respectively). Additionally, both the tetanus toxin and the botulinum toxins are dichain proteins composed of a light chain (molecular weight about 50 kD) covalently bound by a single disulfide bond to a heavy chain (molecular weight about 100 kD). Hence, the molecular weight of tetanus toxin and of each of the seven botulinum toxins (non-complexed) is about 150 kD. Furthermore, for both the tetanus toxin and the botulinum toxins, the light chain bears the domain which exhibits intracellular biological (protease) activity, while the heavy chain comprises the receptor binding (immunogenic) and cell membrane translocational domains.

Further, both the tetanus toxin and the botulinum toxins exhibit a high, specific affinity for gangliocide receptors on the surface of presynaptic cholinergic neurons. Receptor mediated endocytosis of tetanus toxin by peripheral cholinergic neurons results in retrograde axonal transport, blocking of the release of inhibitory neurotransmitters from central synapses and a spastic paralysis. Receptor mediated endocytosis of botulinum toxin by peripheral cholinergic neurons results in little if any retrograde transport, inhibition of acetylcholine exocytosis from the intoxicated peripheral motor neurons and a flaccid paralysis.

Finally, the tetanus toxin and the botulinum toxins resemble each other in both biosynthesis and molecular architecture. Thus, there is an overall 34% identity between the protein sequences of tetanus toxin and botulinum toxin type A, and a sequence identity as high as 62% for some functional domains. Binz T. et al., The Complete Sequence of Botulinum Neurotoxin Type A and Comparison with Other Clostridial Neurotoxins, J Biological Chemistry 265(16);9153-9158:1990.

A toxoid is an antigen which can be used to raise antibodies to and thereby vaccinate against the toxin from which the toxoid is derived. Typically, the toxoid comprises the immunogenic fragment of the toxin (i.e. the carboxyl terminal of the heavy chain (designed as H_{C}) of the tetanus toxin or the botulinum toxins) or a toxin rendered biologically inactive, though still immunogenic, by thermal or chemical (i.e. formalin treatment) denaturation or alteration of the native toxin. Thus, unlike the natural toxin, the toxoid derived from the tetanus or botulinum toxin has been derived of its biological activity, that is its ability to act as an intracellular protease and inhibit neuronal exocytosis of acetylcholine.

Controlled release implants for the therapeutic administration of tetanus toxoid to achieve vaccination against tetanus toxin are known. Thus, the tetanus toxoid as a protein vaccine has been administered incorporated into injectable, biodegradable poly(lactide-co-glycolide) (“PLGA”) microspheres. It has been determined that a water content of a lyophilized tetanus toxoid used to make a tetanus toxoid implant above about 10% can result in significant aggregation and inactivation of the tetanus toxoid. See e.g. pages 251-254 of Schwendeman S. P. et al., Peptide, Protein, and Vaccine Delivery From Implantable Polymeric Systems, chapter 12 (pages 229-267) of Park K., Controlled Drug Delivery Challenges and Strategies, American Chemical Society (1997).

Pulsatile tetanus toxoid implants which permit in vivo subcutaneous administration to mammals of four or five discrete doses (i.e. multiple pulses) of tetanus toxoid over a period in excess of 60 days are known. See e.g. Cardamone M., et al., In Vitro Testing of a Pulsatile Delivery System and its In Vivo Application for Immunization Against Tetanus Toxoid, J Controlled Release 47;205-219:1997.

To be fully immunized against tetanus it is believed to be essential for the patient to receive three consecutive doses of this antigen. Work has been carried out to develop a single dose (i.e. multi pulse) tetanus vaccine implant formulation. This has been achieved using PLA and PLGA microspheres which can release the vaccine in a controlled manner. Encapsulation of tetanus toxoid has been carried out using a water-in-oil-in-water solvent extraction and solvent evaporation techniques with a toxoid loading efficiency of greater than about 80%.

Albumin has been used to improve the stability of microsphere encapsulated protein. Thus, tetanus toxoid co-encapsulation with albumin has been shown to increase both the encapsulation efficiency into PLGA 50:50 (lactide:glycolide) microspheres and the immunogenicity of pulsatile release tetanus toxoid. Johansen P., et al., Improving Stability and Release Kinetics of Microencapsulated Tetanus Toxoid by Co-Encapsulation of Additives, Pharm Res 15(7);1103-1110:1998.

Attempts have been made to reduce encapsulated tetanus toxoid inactivation by polymer degradation products by making PLGA and poloxamer 188 (a non-ionic surfactant) blend microspheres through an oil-in-oil extraction process, the poloxamer 188 reportedly acting to prevent interaction between antigen and polymer. Tobio M., et al., A Novel System Based on a Poloxamer/PLGA Blend as a Tetanus Toxoid Delivery Vehicle, Pharm Res 16(5);682-688:1999.

It is known to combine a plurality of discrete sets of tetanus toxoid incorporating microspheres into a single implant, wherein each set of microspheres has a different polymeric composition and hence a different rate of biodegradation, to thereby provide a pulsatile (multiple pulse) release tetanus toxoid implant. Thus, mice have been injected with a 5% lecithin solution (total volume 100 μl/injection) comprising three discrete set of tetanus toxin incorporating biodegradable, polymeric microspheres. The microspheres used were: (1) poly(D,L-lactide-co-glycolide (PLGA) where the lactide and glycolide copolymers were present in a 50:50 ratio; (2) PLGA 75:25 microspheres, and; (3) poly(D,L-lactide) (PLA) 100:0 microspheres. Lecithin was used to disperse the microspheres. The PLGA 50:50 and the PLGA 75:25 microspheres both showed an initial burst release (over one day) of between 30-40% of the total dose of tetanus toxoid. The remaining tetanus toxoid was delivered between 3-5 weeks after injection from the PLGA 50:50 microspheres and between 8-12 weeks for the PLGA 75:25 microspheres. The PLA 100:0 microspheres did not give an initial burst release, but rather a release of the tetanus toxoid antigen over 4-6 months. Thus, use of a single injection of a mixture of three different tetanus toxoid incorporating microspheres provided four pulses of the tetanus toxoid over a six month period: a first pulse due to the day one burst, a second pulse during weeks 3-5, a third pulse during weeks 8-12 and a fourth pulse during months 4-6. Men Y., et al., G., A Single Administration of Tetanus Toxoid in Biodegradable Microspheres Elicits T Cell and Antibody Responses Similar or Superior to Those Obtained with Aluminum Hydroxide, Vaccine 13, 683-689:1995.

Tetanus and botulinum toxoid vaccines have been made by treating the native toxin with formalin. The U.S. Center for Disease Control can supply a pentavalent, formalin-inactivated toxoid of botulinum toxin types A, B, C, D and E. The pre-exposure immunization schedule calls for subcutaneous administration of the botulinum toxoid vaccine in three dosings at 0, 2 and 12 weeks with a boaster at plus 12 months and yearly boasters at yearly intervals thereafter if antibody levels fall.

U.S. Pat. No. 5,980,948 discusses use of polyetherester copolymer microspheres for encapsulation and controlled delivery of a variety of protein drugs, including tetanus and botulinum antitoxins.

U.S. Pat. No. 5,902,565 discusses A controlled or delayed-release preparation comprising microspherical particles comprising a continuous matrix of biodegradable polymer containing discrete, immunogen-containing regions, where the immunogens can be botulinum toxin type C and D toxoids.

A biodegradable implant for delivering a therapeutic botulinum toxin to an ocular region could provide significant medical benefit for patients afflicted with a medical condition of the eye.

What is needed therefore is a biocompatible botulinum toxin delivery system by which therapeutic amounts of the botulinum toxin can be administered to a human eye.

### SUMMARY

The present invention meets this need and provides a biocompatible, botulinum toxin delivery system by which therapeutic amounts of the botulinum toxin can be administered to a human eye.

The present invention provides a botulinum toxin implant which overcomes the known problems, difficulties and deficiencies associated with topical or oral administration or repeat intravitreal injection of a pharmaceutical, such as an anti-inflammatory drug, to treat an ocular medical condition.

The biodegradable implants and methods of this invention are typically used to treat medical conditions of the eye. Consequently, the implants are sized such that they are appropriate for implantation in the eye of a patient. Preferably, an implant within the scope of the invention disclosed herein is sized for implantation into or on an avascular region of the choroid, such as the pars plana. More preferably, an implant within the scope of the invention disclosed herein is sized for implantation into the vitreous chamber of a patient's eye.

In one embodiment, an implant for treating an ocular disease includes a botulinum toxin dispersed within a biodegradable polymer matrix, wherein the bioerodible implant has an in vivo cumulative release profile in which less than about 15 percent of the botulinum toxin is released about one day after implantation of the bioerodible implant and greater than about 80 percent of the botulinum toxin is released about 28 days after implantation of the bioerodible implant. The biodegradable polymer matrix can comprise a mixture of hydrophilic end group PLGA and hydrophobic end group PLGA.

In another embodiment, a bioerodible implant for treating medical conditions of the eye includes an botulinum toxin dispersed within a biodegradable polymer matrix, wherein the bioerodible implant is formed by an extrusion method, and wherein the bioerodible implant has an in vivo eye cumulative release profile in which greater than about 80 percent of the botulinum toxin is released about 28 days after implantation of the bioerodible implant.

In a further variation, the bioerodible implant for treating medical conditions of the eye includes an botulinum toxin dispersed within a biodegradable polymer matrix, wherein the bioerodible implant exhibits a cumulative release profile in which greater than about 80 percent of the botulinum toxin is released about 28 days after implantation of the bioerodible implant, and wherein the cumulative release profile is approximately sigmoidal in shape over about 28 days after implantation.

In yet a further variation, the bioerodible implant for treating medical conditions of the eye includes an botulinum toxin dispersed within a biodegradable polymer matrix, wherein the biodegradable polymer matrix comprises a mixture of PLGA having hydrophilic end groups and PLGA having hydrophobic end groups. Examples of hydrophilic end groups include, but are not limited to, carboxyl, hydroxyl, and polyethylene glycol. Examples of hydrophobic end groups include, but are not limited to, alkyl esters and aromatic esters.

In yet another variation, the bioerodible implant for treating medical conditions of the eye includes an botulinum toxin dispersed within a biodegradable polymer matrix, wherein the bioerodible implant has an in vivo eye cumulative release profile in which less than about 15 percent of the botulinum toxin is released about one day after implantation of the bioerodible implant and greater than about 80 percent of the botulinum toxin is released about 28 days after implantation of the bioerodible implant.

Botulinum toxins suitable for incorporation into the bioerodible implants of the present invention are the botulinum neurotoxin serotypes A, B, C, D, E, F and G. The implants may be used to treat ocular diseases of human patients. Examples of such ocular diseases include, but are not limited to, uveitis, macular edema, macular degeneration, retinal detachment, ocular tumors, fungal or viral infections, multifocal choroiditis, diabetic retinopathy, proliferative vitreoretinopathy (PVR), sympathetic opthalmia, Vogt Koyanagi-Harada (VKH) syndrome, histoplasmosis, uveal diffusion, vascular occlusion, and the like.

Furthermore, upon implantation in an eye of a patient, the bioerodible implants can deliver the botulinum toxin such that the resulting concentration of botulinum toxin in vivo in the aqueous humor can be approximately 10-fold less than in the vitreous humor. The botulinum toxin can be delivered so that a therapeutic amount of botulinum toxin is provided in the eye region of interest. In general, the therapeutic amount of botulinum toxin in an ocular region can be modified by varying the size of the bioerodible implant.

Also within the scope of the invention disclosed herein is a botulinum toxin ocular delivery system comprising a carrier material and a botulinum toxin associated with the carrier. The toxin can be associated with the carrier by being mixed with and encapsulated by the carrier to thereby form an ocular botulinum toxin delivery system, that is a botulinum toxin ocular implant. The implant can release therapeutic amounts of the botulinum toxin from the carrier in a monophasic manner or as a plurality of pulses in vivo upon intravitreal insertion of the implant system into a human eye.

The carrier can comprise a tablet, wafer, sheet, plaque or a plurality of polymeric microspheres (i.e. a polymeric matrix) and substantial amounts of the botulinum toxin has not be transformed into a botulinum toxoid prior to association of the botulinum toxin with the carrier. That is, significant amounts of the botulinum toxin associated with the carrier have a toxicity which is substantially unchanged relative to the toxicity of the botulinum toxin prior to association of the botulinum toxin with the carrier.

According to the present invention, the botulinum toxin can be released from the carrier over of a period of time of from about 1 day to about 6 years and the carrier is preferably comprised of a substance which is substantially biodegradable. The botulinum toxin is one of the botulinum toxin types A, B, C₁, D, E, F and G and is preferably botulinum toxin type A. The botulinum toxin can be associated with the carrier in an amount of between about 1 unit and about 3,000 units of the botulinum toxin. Preferably, the quantity of the botulinum toxin associated with the carrier is between about 1 units and about 50 units of a botulinum toxin type A. Where the botulinum toxin is botulinum toxin type B, preferably, the quantity of the botulinum toxin associated with the carrier is between about 50 units and about 3,000 units of a botulinum toxin type B.

A detailed embodiment of the present invention can comprise a controlled release system, comprising a biodegradable polymer and between about 1 units and about 3,000 units of a botulinum toxin encapsulated by the polymer carrier, thereby forming a controlled release system, wherein therapeutic amounts of the botulinum toxin can be released from the carrier in a pulsatile or non-pulsatile manner in vivo upon intravitreal implantation of the controlled release system in a human eye over a period of time extending from about 1 day to about 6 years.

A method for making an implant within the scope of the present invention can have the steps of: dissolving a polymer in a solvent to form a polymer solution; mixing or dispersing a botulinum toxin in the polymer solution to form a polymer-botulinum toxin mixture, and; allowing the polymer-botulinum toxin mixture to set or cure, thereby making an implant for pulsatile release of the botulinum toxin. This method can have the further step after the mixing step of evaporating solvent.

A method for using a pulsatile release implant within the scope of the present invention can be by injecting or implanting a polymeric implant which includes a botulinum toxin, thereby treating an ocular disease.

An alternate embodiment of the present invention can be a carrier comprising a polymer selected from the group of polymers consisting of polylactides and polyglycolides and a stabilized botulinum toxin associated with the carrier, thereby forming a pulsatile release botulinum toxin delivery system, wherein therapeutic amounts of the botulinum toxin can be released from the carrier in a plurality of pulses in vivo upon intravitreal implantation of the delivery system in a human patient. The carrier can comprise a plurality of discrete sets of polymeric, botulinum toxin incorporating microspheres, wherein each set of polymers has a different polymeric composition.

Definitions

The following definitions apply herein.

“About” means plus or minus ten percent of the value so qualified.

“Biocompatible” means that there is an insignificant inflammatory response at the site of implantation from use of the implant.

“Bioerodible” is synonymous with “biodegradable”. Biodegradable means that the item, material or substance substantially dissolves within one year after placement of the material in a physiological fluid, such as the vitreous.

“Effective amount” as applied to the biologically active compound means that amount of the compound which is generally sufficient to effect a desired change in the subject, for example a reduction in inflammation.

“Implant” means a drug delivery system comprised of a biocompatible polymer or ceramic material which contains or which can act as a carrier for a molecule with a biological activity. The implant can be, injected, inserted or implanted into a human eye.

By “therapeutic amount” it is meant a concentration of botulinum toxin that has been locally delivered to an ocular region that is appropriate to safely treat a medical condition of the eye.

“Cumulative release profile” means the cumulative total percent of botulinum toxin released from the implant either into the posterior segment in vivo in human eyes over time or into the specific release medium in vitro over time.

“Substantially” means between seventy percent to one hundred percent of the item, material, drug or condition to which such an adjective is applied

A method for making an implant within the scope of the present invention for controlled release of a botulinum neurotoxin, can include dissolving a biocompatible polymer in a polymer solvent to form a polymer solution, dispersing particles of biologically active, stabilized neurotoxin in the polymer solution, and then solidifying the polymer to form a polymeric matrix containing a dispersion of the neurotoxin particles.

A method of using an implant within the scope of the present invention forming for controlled release of a neurotoxin can comprise providing a therapeutically effective level of biologically active, botulinum neurotoxin in a patient for a prolonged period of time by implanting the implant into the vitreous chamber of a patient's eye.

Another embodiment of my invention can comprise a botulinum toxin system, comprising a carrier and a botulinum toxin associated with the carrier, thereby forming a botulinum toxin system, wherein a botulinum toxin can be released from the carrier upon implantation of the botulinum system in a human eye.

The carrier can comprise a plurality of polymeric microspheres. Preferably, substantial amounts of the botulinum toxin has not be transformed into a botulinum toxoid prior to association of the botulinum toxin with the carrier. That is, significant amounts of the botulinum toxin associated with the carrier have a toxicity which is substantially unchanged relative to the toxicity of the botulinum toxin prior to association of the botulinum toxin with the carrier.

The carrier can comprise a polymeric matrix and the botulinum toxin can be released from the carrier over of a period of time of from about 10 days to about 6 years. In one embodiment the carrier is comprised of a substance which is substantially biodegradable. As previously set forth, the botulinum toxin can be selected from the group consisting of botulinum toxin types A, B, C₁, D, E, F and G, and preferably the botulinum toxin is a botulinum toxin type A.

### DESCRIPTION

The present invention provides biodegradable ocular implants and methods for treating medical conditions of the eye (i.e. an ocular disease). Usually, the implants are formed to be monolithic, i.e., the botulinum neuro toxin is distributed throughout the biodegradable polymer matrix. Furthermore, the implants are formed to release an botulinum toxin into the vitreous chamber of the eye over various time periods. The botulinum toxin may be release over a time period including, but is not limited to, approximately six months, approximately three months, approximately one month, or less than one month.

Biodegradable Implants

The implants of the invention include an botulinum toxin dispersed within a biodegradable polymer. The implant compositions typically vary according to the preferred drug release profile, the particular botulinum toxin used, the condition being treated, and the medical history of the patient. Botulinum toxins that may be used include, but are not limited to, the botulinum neurotoxin serotypes A, B, C, D, E, F and G. In one embodiment, the biodegradable implant includes a combination of two or more botulinum neurotoxins.

The botulinum toxin can constitute from about 10% to about 90% by weight of the implant. In one variation, the botulinum toxin is from about 40% to about 80% by weight of the implant. In a preferred variation, the botulinum toxin comprises about 60% by weight of the implant.

In one variation, the botulinum toxin may be homogeneously dispersed in the biodegradable polymer matrix of the implants. The selection of the biodegradable polymer matrix to be employed will vary with the desired release kinetics, patient tolerance, the nature of the disease to be treated, and the like. Polymer characteristics that are considered include, but are not limited to, the biocompatibility and biodegradability at the site of implantation, compatibility with the botulinum toxin of interest, and processing temperatures. The biodegradable polymer matrix usually comprises at least about 10, at least about 20, at least about 30, at least about 40, at least about 50, at least about 60, at least about 70, at least about 80, or at least about 90 weight percent of the implant. In one variation, the biodegradable polymer matrix comprises about 40% by weight of the implant.

Biodegradable polymer matrices which may be employed include, but are not limited to, polymers made of monomers such as organic esters or ethers, which when degraded result in physiologically acceptable degradation products. Anhydrides, amides, orthoesters, or the like, by themselves or in combination with other monomers, may also be used. The polymers are generally condensation polymers. The polymers may be crosslinked or non-crosslinked. If crosslinked, they are usually not more than lightly crosslinked, and are less than 5% crosslinked, usually less than 1% crosslinked.

For the most part, besides carbon and hydrogen, the polymers will include oxygen and nitrogen, particularly oxygen. The oxygen may be present as oxy, e.g., hydroxy or ether, carbonyl, e.g., non-oxo-carbonyl, such as carboxylic acid ester, and the like. The nitrogen may be present as amide, cyano, and amino. An exemplary list of biodegradable polymers that may be used are described in Heller, Biodegradable Polymers in Controlled Drug Delivery, In: “CRC Critical Reviews in Therapeutic Drug Carrier Systems”, Vol. 1. CRC Press, Boca Raton, Fla. (1987).

Of particular interest are polymers of hydroxyaliphatic carboxylic acids, either homo- or copolymers, and polysaccharides. Included among the polyesters of interest are homo- or copolymers of D-lactic acid, L-lactic acid, racemic lactic acid, glycolic acid, caprolactone, and combinations thereof. Copolymers of glycolic and lactic acid are of particular interest, where the rate of biodegradation is controlled by the ratio of glycolic to lactic acid. The percent of each monomer in poly(lactic-co-glycolic)acid (PLGA) copolymer may be 0-100%, about 15-85%, about 25-75%, or about 35-65%. In a preferred variation, a 50/50 PLGA copolymer is used. More preferably, a random copolymer of 50/50 PLGA is used.

Biodegradable polymer matrices that include mixtures of hydrophilic and hydrophobic ended PLGA may also be employed, and are useful in modulating polymer matrix degradation rates. Hydrophobic ended (also referred to as capped or end-capped) PLGA has an ester linkage hydrophobic in nature at the polymer terminus. Typical hydrophobic end groups include, but are not limited to alkyl esters and aromatic esters. Hydrophilic ended (also referred to as uncapped) PLGA has an end group hydrophilic in nature at the polymer terminus. PLGA with a hydrophilic end groups at the polymer terminus degrades faster than hydrophobic ended PLGA because it takes up water and undergoes hydrolysis at a faster rate (Tracy et al., Biomaterials 20:1057-1062 (1999)). Examples of suitable hydrophilic end groups that may be incorporated to enhance hydrolysis include, but are not limited to, carboxyl, hydroxyl, and polyethylene glycol. The specific end group will typically result from the initiator employed in the polymerization process. For example, if the initiator is water or carboxylic acid, the resulting end groups will be carboxyl and hydroxyl. Similarly, if the initiator is a monofunctional alcohol, the resulting end groups will be ester or hydroxyl.

The implants may be formed from all hydrophilic end PLGA or all hydrophobic end PLGA. In general, however, the ratio of hydrophilic end to hydrophobic end PLGA in the biodegradable polymer matrices of this invention range from about 10:1 to about 1:10 by weight. For example, the ratio may be 3:1, 2:1, or 1:1 by weight. In a preferred variation, an implant with a ratio of hydrophilic end to hydrophobic end PLGA of 3:1 w/w is used.

Other agents may be employed in the formulation for a variety of purposes. For example, buffering agents and preservatives may be employed. Preservatives which may be used include, but are not limited to, sodium bisulfite, sodium bisulfate, sodium thiosulfate, benzalkonium chloride, chlorobutanol, thimerosal, phenylmercuric acetate, phenylmercuric nitrate, methylparaben, polyvinyl alcohol and phenylethyl alcohol. Examples of buffering agents that may be employed include, but are not limited to, sodium carbonate, sodium borate, sodium phosphate, sodium acetate, sodium bicarbonate, and the like, as approved by the FDA for the desired route of administration. Electrolytes such as sodium chloride and potassium chloride may also be included in the formulation.

The biodegradable ocular implants may also include additional hydrophilic or hydrophobic compounds that accelerate or retard release of the botulinum toxin. Furthermore, the inventors believe that because hydrophilic end PLGA has a higher degradation rate than hydrophobic end PLGA due to its ability to take up water more readily, increasing the amount of hydrophilic end PLGA in the implant polymer matrix will result in faster dissolution rates. The time from implantation to significant release of botulinum toxin (lag time) can increase with decreasing amounts of hydrophilic end PLGA in the ocular implant. Thus, the lag time for implants having 0% hydrophilic end PLGA (40% w/w hydrophobic end) can be about 21 days. In comparison, a significant reduction in lag time can be seen with implants having 10% w/w and 20% w/w hydrophilic end PLGA.

It is believed that release of the botulinum toxin is achieved by erosion of the biodegradable polymer matrix and by diffusion of the botulinum toxin into an ocular fluid, e.g., the vitreous, with subsequent dissolution of the polymer matrix and release of the botulinum toxin. The actors that influence the release kinetics include such characteristics as the size of the botulinum toxin, the solubility of the botulinum toxin, the ratio of botulinum toxin to polymer(s), the method of manufacture, the surface area exposed, and the erosion rate of the polymer(s). The release kinetics achieved by this form of botulinum toxin release are different than that achieved through formulations which release botulinum toxins through polymer swelling, such as with crosslinked hydrogels. In that case, the botulinum toxin is not released through polymer erosion, but through polymer swelling, which releases botulinum toxin as liquid diffuses through the pathways exposed.

Additionally, the release rate of the botulinum toxin depends at least in part on the rate of degradation of the polymer backbone component or components making up the biodegradable polymer matrix. For example, condensation polymers may be degraded by hydrolysis (among other mechanisms) and therefore any change in the composition of the implant that enhances water uptake by the implant will likely increase the rate of hydrolysis, thereby increasing the rate of polymer degradation and erosion, and thus increasing the rate of botulinum toxin release.

The release kinetics of the implants of the invention are dependent in part on the surface area of the implants. A larger surface area exposes more polymer and botulinum toxin to ocular fluid, causing faster erosion of the polymer matrix and dissolution of the botulinum toxin particles in the fluid. The size and shape of the implant may also be used to control the rate of release, period of treatment, and botulinum toxin concentration at the site of implantation. At equal botulinum toxin loads, larger implants will deliver a proportionately larger dose, but depending on the surface to mass ratio, may possess a slower release rate. For implantation in an ocular region, the total weight of the implant preferably ranges, e.g., from about 100-5000 μg, usually from about 500-1500 μg. In one variation, the total weight of the implant is about 600 μg. In another variation, the total weight of the implant is about 1200 μg.

The bioerodible implants are typically solid, and may be formed as particles, sheets, patches, plaques, films, discs, fibers, rods, and the like, or may be of any size or shape compatible with the selected site of implantation, as long as the implants have the desired release kinetics and deliver an amount of botulinum toxin that is therapeutic for the intended medical condition of the eye. The upper limit for the implant size will be determined by factors such as the desired release kinetics, toleration for the implant at the site of implantation, size limitations on insertion, and ease of handling. For example, the vitreous chamber is able to accommodate relatively large rod-shaped implants, generally having diameters of about 0.05 mm to 3 mm and a length of about 0.5 to about 10 mm. In one variation, the rods have diameters of about 0.1 mm to about 1 mm. In another variation, the rods have diameters of about 0.3 mm to about 0.75 mm. In yet a further variation, other implants having variable geometries but approximately similar volumes may also be used.

As previously discussed, the release of an botulinum toxin from a biodegradable polymer matrix may also be modulated by varying the ratio of hydrophilic end PLGA to hydrophobic end PLGA in the matrix. Release rates may be further manipulated by the method used to manufacture the implant. For instance, extruded 60/40 w/w botulinum toxin/PLGA implants having a ratio of hydrophilic end and hydrophobic end PLGA of 3:1, compared to compressed tablet implants, can demonstrate a different drug release profile and concentration of the botulinum toxin in the vitreous over about a one month period. Overall, a lower burst of botulinum toxin release and a more consistent level of botulinum toxin in the vitreous can be demonstrated with the extruded implants.

The proportions of botulinum toxin, biodegradable polymer matrix, and any other additives may be empirically determined by formulating several implants with varying proportions and determining the release profile in vitro or in vivo. A USP approved method for dissolution or release test can be used to measure the rate of release in vitro (USP 24; NF 19 (2000) pp. 1941-1951). For example, a weighed sample of the implant is added to a measured volume of a solution containing 0.9% NaCl in water, where the solution volume will be such that the botulinum toxin concentration after release is less than 20% of saturation. The mixture is maintained at 37° C. and stirred or shaken slowly to maintain the implants in suspension. The release of the dissolved botulinum toxin as a function of time may then be followed by various methods known in the art, such as spectrophotometrically, HPLC, mass spectroscopy, and the like, until the solution concentration becomes constant or until greater than 90% of the botulinum toxin has been released.

In one variation, the extruded implants described herewith (ratio of hydrophilic end PLGA to hydrophobic end PLGA of 3:1) may have in vivo cumulative percentage release profiles with the following described characteristics. At day one after implantation, the percentage in vivo cumulative release may be between about 0% and about 15%, and more usually between about 0% and about 10%. At day one after implantation, the percentage in vivo cumulative release may be less than about 15%, and more usually less than about 10%.

At day three after implantation, the percentage in vivo cumulative release may be between about 0% and about 20%, and more usually between about 5% and about 15%. At day three after implantation, the percentage in vivo cumulative release may be less than about 20%, and more usually less than about 15%.

At day seven after implantation, the percentage in vivo cumulative release may be between about 0% and about 35%, more usually between about 5% and about 30%, and more usually still between about 10% and about 25%. At day seven after implantation, the percentage in vivo cumulative release may be greater than about 2%, more usually greater than about 5%, and more usually still greater than about 10%.

At day fourteen after implantation, the percentage in vivo cumulative release may be between about 20% and about 60%, more usually between about 25% and about 55%, and more usually still between about 30% and about 50%. At day fourteen after implantation, the percentage in vivo cumulative release may be greater than about 20%, more usually greater than about 25%, and more usually still greater than about 30%.

At day twenty-one after implantation, the percentage in vivo cumulative release may be between about 55% and about 95%, more usually between about 60% and about 90%, and more usually still between about 65% and about 85%. At day twenty-one after implantation, the percentage in vivo cumulative release may be greater than about 55%, more usually greater than about 60%, and more usually still greater than about 65%.

At day twenty-eight after implantation, the percentage in vivo cumulative release may be between about 80% and about 100%, more usually between about 85% and about 100%, and more usually still between about 90% and about 100%. At day twenty-eight after implantation, the percentage in vivo cumulative release may be greater than about 80%, more usually greater than about 85%, and more usually still greater than about 90%.

At day thirty-five after implantation, the percentage in vivo cumulative release may be between about 95% and about 100%, and more usually between about 97% and about 100%. At day thirty-five after implantation, the percentage in vivo cumulative release may be greater than about 95%, and more usually greater than about 97%.

In one variation, the percentage in vivo cumulative release has the following characteristics: one day after implantation it is less than about 15%; three days after implantation it is less than about 20%; seven days after implantation it is greater than about 5%; fourteen days after implantation it is greater than about 25%; twenty-one days after implantation it is greater than about 60%; and twenty-eight days after implantation it is greater than about 80%. In another variation, the percentage in vivo cumulative release has the following characteristics: one day after implantation it is less than about 10%; three days after implantation it is less than about 15%; seven days after implantation it is greater than about 10%; fourteen days after implantation it is greater than about 30%; twenty-one days after implantation it is greater than about 65%; twenty-eight days after implantation it is greater than about 85%.

In yet another variation, the extruded implants described herein may have in vitro cumulative percentage botulinum neurotoxin release profiles in saline solution at 37° C. with the following characteristics. The percentage in vitro cumulative release at day one may be between about 0% and about 5%, and more usually between about 0% and about 3%. The percentage in vitro cumulative release at day one may be less than about 5%, and more usually less than about 3%.

The percentage in vitro cumulative release at day four may be between about 0% and about 7%, and more usually between about 0% and about 5%. The percentage in vitro cumulative release at day four may be less than about 7%, and more usually less than about 5%.

The percentage in vitro cumulative release at day seven may be between about 1% and about 10%, and more usually between about 2% and about 8%. The percentage in vitro cumulative release at day seven may be greater than about 1%, and more usually greater than about 2%.

The percentage in vitro cumulative release at day 14 may be between about 25% and about 65%, more usually between about 30% and about 60%, and more usually still between about 35% and about 55%. The percentage in vitro cumulative release at day 14 may be greater than about 25%, more usually greater than about 30%, and more usually still greater than about 35%.

The percentage in vitro cumulative release at day 21 may be between about 60% and about 100%, more usually between about 65% and about 95%, and more usually still between about 70% and about 90%. The percentage in vitro cumulative release at day 21 may be greater than about 60%, more usually greater than about 65%, and more usually still greater than about 70%.

The percentage in vitro cumulative release at day 28 may be between about 75% and about 100%, more usually between about 80% and about 100%, and more usually still between about 85% and about 95%. The percentage in vitro cumulative release at day 28 may be greater than about 75%, more usually greater than about 80%, and more usually still greater than about 85%.

The percentage in vitro cumulative release at day 35 may be between about 85% and about 100%, more usually between about 90% and about 100%, and more usually still between about 95% and about 100%. The percentage in vitro cumulative release at day 35 may be greater than about 85%, more usually greater than about 90%, and more usually still greater than about 95%.

In one variation, the percentage in vitro cumulative release has the following characteristics: after one day it is less than about 1%; after four days it is less than about 7%; after seven days it is greater than about 2%; after 14 days it is greater than about 30%; after 21 days it is greater than about 65%; after 28 days it is greater than about 80%; and after 35 days it is greater than about 90%. In another variation, the percentage in vitro cumulative release has the following characteristics: after one day it is less than about 3%; after four days it is less than about 5%; after seven days it is greater than about 2%; after 14 days it is greater than about 35%; after 21 days it is greater than about 70%; after 28 days it is greater than about 85%; and after 35 days it is greater than about 90%.

Besides showing a lower burst effect for the extruded implants, after 28 days in vivo in human eyes, or in vitro in a saline solution at 37° C., respectively, almost all of the botulinum toxin can be released from the implants. Furthermore, the botulinum toxin release profiles for the extruded implants in vivo (from the time of implantation) and in vitro (from the time of placement into a saline solution at 37° C.) can be substantially similar and follow approximately a sigmoidal curve, releasing substantially all of the botulinum toxin over 28 days. From day one to approximately day 17, the curves can show approximately an upward curvature (i.e., the derivative of the curve increases as time increases), and from approximately day 17 onwards the curves can show approximately a downward curvature (i.e., the derivative of the curve decreases as time increases).

In contrast, botulinum toxin compressed tablet implants can exhibit a higher initial burst of botulinum toxin release generally followed by a gradual increase in release. Furthermore, implantation of a compressed implant can result in different concentrations of botulinum toxin in the vitreous at various time points from implants that have been extruded. For example, with extruded implants there can be a gradual increase, plateau, and gradual decrease in intravitreal botulinum toxin concentrations. In contrast, for compressed tablet implants, there can be a higher initial botulinum toxin release followed by an approximately constant decrease over time. Consequently, the intravitreal concentration curve for extruded implants can result in more sustained levels of botulinum toxin in the ocular region.

In addition to the previously described implants releasing substantially all of the therapeutic botulinum toxin within 35 days, by varying implant components including, but not limited to, the composition of the biodegradable polymer matrix, implants can also be formulated to release a therapeutic botulinum toxin for any desirable duration of time, for example, for about one week, for about two weeks, for about three weeks, for about four weeks, for about five weeks, for about six weeks, for about seven weeks, for about eight weeks, for about nine weeks, for about ten weeks, for about eleven weeks, for about twelve weeks, or for more than 12 weeks.

Another important feature of the extruded implants is that different concentration levels of botulinum toxin may be established in the vitreous using different doses of the botulinum toxin. The concentration of botulinum toxin in the vitreous can be significantly larger with a 10 unit botulinum toxin type A (BOTOX®) extruded implant than with the 5 unit botulinum toxin type A (BOTOX®) extruded implant. Different botulinum toxin concentrations are not demonstrated with the compressed tablet implant. Thus, by using an extruded implant, it is possible to more easily control the concentration of botulinum toxin in the vitreous. In particular, specific dose-response relationships may be established since the implants can be sized to deliver a predetermined amount of botulinum toxin.

To reiterate, the present invention is based upon the discovery that a botulinum toxin ocular implant can be made comprising a carrier and a botulinum toxin associated with the carrier. The botulinum toxin system can be implanted intraocularly in a human patient and therapeutically effective amounts of the botulinum toxin can be released from the carrier into the vitreous chamber.

Without being bound by theory, a mechanism can be postulated for the efficacy of the invention disclosed herein. Thus, there is considerable evidence that botulinum toxin can act to reduce inflammation. See e.g. Silberstein S. et al., Botulinum toxin type A: Myths, facts, and current research, Headache 2003 July;43 Suppl 1 1 (Suppl 1):S1; Cui, M. et al., Botulinum toxin type A (BTX-A) reduces inflammatory pain in the rat formalin model, Cephalagia 2000;20(4):414, and U.S. Pat. No. 6,063,768 (treatment of neurogenic inflammation with a botulinum toxin). It can therefore be expected that the botulinum toxin ocular implants disclosed herein can be used to treat ocular conditions, such as intraocular inflammation conditions.

In one embodiment of the botulinum toxin delivery system within the scope of the present invention, the system is capable of pulsatile (i.e. multiphasic) release of therapeutic amounts of a botulinum toxin. By pulsatile release it is meant that during a period of time, which can extend from about 1 hour to about 4 weeks, a quantity of therapeutically effective (i.e. biologically active) botulinum toxin is released from a carrier material in vivo at the site of implantation. The pulse of released botulinum toxin can comprise (for a botulinum toxin type A) as little as about 1 unit to as much as 200 units. The quantity of botulinum toxin required for therapeutic efficacy can be varied according to the known clinical potency of the different botulinum toxin serotypes. For example, several orders of magnitude more units of a botulinum toxin type B are typically required to achieve a physiological effect comparable to that achieved from use of a botulinum toxin type A. Prior to and following each pulse there is a period of reduced or substantially no botulinum toxin release from the implant.

The botulinum toxin released in therapeutically effective amounts by a controlled release delivery system within the scope of the present invention is preferably, substantially biologically active botulinum toxin. In other words, the botulinum toxin released from the disclosed delivery system is capable of binding with high affinity to a cholinergic neuron, being translocated, at least in part, across the neuronal membrane, and through its activity in the cytosol of the neuron of inhibiting exocytosis of acetylcholine from the neuron. The present invention excludes from its scope use deliberate use of a botulinum toxoid as an antigen in order to confer immunity to the botulinum toxin through development of antibodies (immune response) due to the immunogenicity of the toxoid.

Pulsatile release of a botulinum toxin from an implant can be accomplished by preparing a plurality of implants with differing carrier material compositions. For example, holding other factors, such as polymer molecular weight, constant an implant can be made up of a several sets of botulinum toxin encapsulated microspheres, each set of microspheres having a different polymer composition such that the polymers of each set of microspheres degrade, and release toxin, at differing rates. Conveniently, the plurality of sets of differing polymer composition microspheres can be pressed into the form of a disc, and implanted as a pellet. The pulsatile release implant can be implanted intravitreally.

A first pulse of a botulinum toxin can be locally administered due to the presence of a botulinum toxin (i.e. free or non-implant incorporated botulinum toxin) administered in conjunction with and at the same time as insertion of the implant and/or due to a burst effect of botulinum toxin release from the implanted microspheres. A second pulse of a botulinum toxin can be administered by the implant at about three months post implantation upon biodegradation of a first set of microspheres. A third pulse of a botulinum toxin can be delivered by the system at about six months post implantation upon dissolution of a second set of bioerodible microspheres, and so on. Thus, a botulinum toxin delivery system within the scope of the present invention which comprises three differing sets of appropriate microsphere polymer compositions, permits a patient to be reimplanted or reinvested with a botulinum toxin only once every 12 months.

For example, it is known that biodegradable PLA:PGA microspheres can be made with varying copolymer content such that proportionally different polymer degradation time windows result. Thus, a 75:25 lactide:glycolide polymer can degrade at about ninety days post implantation. Additionally, a 100:0 lactide:glycolide polymer can degrade at about one hundred and eighty days post implantation. Furthermore, a 95:5 poly(DL-lactide):glycolide polymer can degrade at about two hindered and seventy days post implantation. Finally, a 100:0 poly(DL-lactide):glycolide polymer can degrade at about twelve months post implantation. See e.g. Kissel et al, Microencapsulation of Antigens Using Biodegradable Polymers: Facts and Fantasies, Behring Inst. Mitt., 98;172-183:1997; Cleland J. L., et al, Development of a Single-Shot Subunit Vaccine for HOV-1: Part 4. Optimizing Microencapsulation and Pulsatile Release of MN rpg120 from Biodegradable Microspheres, J Cont Rel 47;135-150:1997, and; Lewis D. H., Controlled Release of Bioagents from Lactide/Glycolide Polymers, pages 1-41 of Chasin M., et al, “Biodegradable Polymers as Drug-Delivery Systems”, Marcel Dekker, New York (1990). The above-specified four discrete sets of polymeric microspheres can be prepared as botulinum toxin incorporating microspheres, and combined into a single implant capable of pulsatile release of the botulinum toxin over a one year period, thereby providing a patient treatment period per implant of about 15-16 months.

The delivery system is prepared so that the botulinum toxin is substantially uniformly dispersed in a biodegradable carrier. An alternate pulsatile delivery system within the scope of the present invention can comprise a carrier coated by a biodegradable coating, either the thickness of the coating or the coating material being varied, such that in the different sets of microspheres, the respective coating take from 3, 6, 9, etc months to be dissolved, thereby providing the desired toxin pulses. The microspheres are inert and are of such a size or due to being pressed into a disc, that they do no diffuse significantly beyond the site of injection. Hence, multiple implantations, as by needle injection, can be carried out at the same time.

A third embodiment within the scope of the present invention of a pulsatile, implant can comprise a non-porous, non-biodegradable, biocompatible tube which is closed at one end. Carrier associated neurotoxin is interspaced discrete locations within the bore of the tube. Thus, toxin at an open or porous, or erodible plug sealed pug the end of the tube rapidly diffuses out, causing the first local administration. Toxin further from the end of the tube takes longer to diffuse out and results in the second local

The thickness of the implant can be used to control the absorption of water by, and thus the rate of release of a neurotoxin from, a composition of the invention, thicker implants releasing the polypeptide more slowly than thinner ones.

The botulinum neurotoxin can be mixed with other excipients, such as bulking botulinum toxins or additional stabilizing botulinum toxins, such as buffers to stabilize the neurotoxin during lyophilization.

The carrier is preferably comprised of a non-toxic, non-immunological, biocompatible material. Suitable the implant materials can include polymers of poly(2-hydroxy ethyl methacrylate) (p-HEMA), poly(N-vinyl pyrrolidone) (p-NVP)+, poly(vinyl alcohol) (PVA), poly(acrylic acid) (PAA), polydimethyl siloxanes (PDMS), ethylene-vinyl acetate copolymers (EVAc), a polymethylmethacrylate (PMMA), polyvinylpyrrolidone/methylacrylate copolymers, poly(lactic acid) (PLA), poly(glycolic acid) (PGA), polyanhydrides, poly(ortho esters), collagen and cellulosic derivatives and bioceramics, such as hydroxyapatite (HPA), tricalcium phosphate (TCP), and aliminocalcium phosphate (ALCAP).

Biodegradable carriers can be made from polymers of poly(lactides), poly(glycolides), collagens, poly(lactide-co-glycolides), poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polycyanoacrylates, poly(p-dioxanone), poly(alkylene oxalates), biodegradable polyurethanes, blends and copolymers thereof. Particularly preferred carriers are formed as polymers or copolymers of poly(lactic-co-glycolic acid) (“PLGA”), where the lactide:glycolide ratio can be varied depending on the desired carrier degradation rate.

Biodegradable PLGA polymers have been used to form resorbable sutures and bone plates and in several commercial microparticle formulations. PLGA degrades through bulk erosion to produce lactic and glycolic acid and is commercially available in a variety of molecular weight and polymer end groups (e.g. lauryl alcohol or free acid). Polyanhydrides are another group of polymers that have been approved for use I humans, and have been used to deliver proteins and antigens. Unlike PLGA, polyanhydrides degrade by surface erosion, releasing botulinum neurotoxin entrapped at the carrier surface.

To prepare a suitable implant, the carrier polymer can be dissolved in an organic solvent such as methylene chloride or ethyl acetate and the botulinum toxin is then mixed into the polymer solution. The conventional processes for microsphere formation are solvent evaporation and solvent (coacervation) methods. The water-in-oil-in-water (W/O/W) double emulsion method is a widely used method of protein antigen encapsulation into PLGA microspheres.

An aqueous solution of a botulinum toxin can be used to make a pulsatile implant. An aqueous solution of the neurotoxin is added to the polymer solution (polymer previously dissolved in a suitable organic solvent). The volume of the aqueous (neurotoxin) solution relative to the volume of organic (polymer) solvent is an important parameter in the determination of both the release characteristics of the microspheres and with regard to the encapsulation efficiency (ratio of theoretical to experimental protein loading) of the neurotoxin.

The encapsulation efficiency can also be increased by increasing the kinematic viscosity of the polymer solution. The kinematic viscosity of the polymer solution can be increased by decreasing the operating temperature and/or by increasing the polymer concentration in the organic solvent.

Thus, with a low aqueous phase (neurotoxin) to organic phase (polymer) volume ratio (i.e. aqueous volume:organic volume is <0.1 ml/ml) essentially 100% of the neurotoxin can be encapsulated by the microspheres and the microspheres can show a triphasic release: an initial burst (first pulse), a lag phase with little or no neurotoxin being released and a second release phase (second pulse).

The length of the lag phase is dependent upon the polymer degradation rate which is in turn dependant upon polymer composition and molecular weight. Thus, the lag phase between the first (burst) pulse and the second pulse increases as the lactide content is increased, or as the polymer molecular weight is increased with the lactide:glycolide ratio being held constant. In addition to a low aqueous phase (neurotoxin) volume, operation at low temperature (2-8 degrees C.), as set forth above, increases the encapsulation efficiency, as well as reducing the initial burst and promoting increased neurotoxin stability against thermal inactivation

Suitable implants within the scope of the present invention for the controlled in vivo release of a neurotoxin, such as a botulinum toxin, can be prepared so that the implant releases the neurotoxin in a pulsatile manner. A pulsatile release implant can release a neurotoxin is a biphasic or multiphase manner. Thus, a pulsatile release implant can have a relatively short initial induction (burst) period, followed by periods during which reduced, little or no neurotoxin is released.

A controlled release of biologically active neurotoxin is a release which results in therapeutically effective, with negligible serum levels, of biologically active, neurotoxin over a period longer than that obtained following direct administration of aqueous neurotoxin. It is preferred that a controlled release be a release of neurotoxin for a period of about six months or more, and more preferably for a period of about one year or more.

An implant within the scope of the present invention can also be formulated as a suspension for injection. Such suspensions may be manufactured by general techniques well known in the pharmaceutical art, for example by milling the polylactide/polypeptide mixture in an ultracentrifuge mill fitted with a suitable mesh screen, for example a 120 mesh, and suspending the milled, screened particles in a solvent for injection, for example propylene glycol, water optionally with a conventional viscosity increasing or suspending botulinum toxin, oils or other known, suitable liquid vehicles for injection.

Denaturation of the encapsulated neurotoxin in the body at 37 degrees C. for a prolonged period of time can be reduced by stabilizing the neurotoxin by lyophilizing it with albumin, lyophilizing from an acidic solution, lyophilizing from a low moisture content solution (these three criteria can be met with regard to a botulinum toxin type A by use of non-reconstituted Botox®) and using a specific polymer matrix composition.

Preferably, the release of biologically active neurotoxin in vivo does not result in a significant immune system response during the release period of the neurotoxin.

A pulsatile botulinum toxin delivery system preferably permits botulinum release from biodegradable polymer microspheres in a biologically active form, that is with a substantially native toxin conformation. To stabilize a neurotoxin, both in a format which renders the neurotoxin useful for mixing with a suitable polymer which can form the implant matrix (i.e. a powdered neurotoxin which has been freeze dried or lyophilized) as well as while the neurotoxin is present or incorporated into the matrix of the selected polymer, various pharmaceutical excipients can be used. Suitable excipients can include starch, cellulose, talc, glucose, lactose, sucrose, gelatin, malt, rice, flour, chalk, silica gel, magnesium stearate, sodium stearate, glycerol monostearate, sodium chloride, albumin and dried skim milk. The neurotoxin in a neurotoxin controlled release composition can be mixed with excipients, bulking botulinum toxins and stabilizing botulinum toxins, and buffers to stabilize the neurotoxin during lyophilization or freeze drying.

It has been discovered that a stabilized neurotoxin can comprise biologically active, non-aggregated neurotoxin complexed with at least one type of multivalent metal cation which has a valiancy of +2 or more.

Suitable multivalent metal cations include metal cations contained in biocompatible metal cation components. A metal cation component is biocompatible if the cation component is non-toxic to the recipient, in the quantities used, and also presents no significant deleterious or untoward effects on the recipient's body, such as an immunological reaction at the injection site.

Preferably, the molar ratio of metal cation component to neurotoxin, for the metal cation stabilizing the neurotoxin, is between about 4:1 to about 100:1 and more typically about 4:1 to about 10:1.

A preferred metal cation used to stabilize a botulinum toxin is Zn⁺⁺ because the botulinum toxin are known to be zinc endopeptidases. Divalent zinc cations are preferred because botulinum toxin is known to be a divalent zinc endopeptidase. In a more preferred embodiment, the molar ratio of metal cation component, containing Zn⁺⁺ cations, to neurotoxin is about 6:1.

The suitability of a metal cation for stabilizing neurotoxin can be determined by one of ordinary skill in the art by performing a variety of stability indicating techniques such as polyacrylamide gel electrophoresis, isoelectric focusing, reverse phase chromatography, HPLC and potency tests on neurotoxin lyophilized particles containing metal cations to determine the potency of the neurotoxin after lyophilization and for the duration of release from microparticles. In stabilized neurotoxin, the tendency of neurotoxin to aggregate within a microparticle during hydration in vivo and/or to lose biological activity or potency due to hydration or due to the process of forming a controlled release composition, or due to the chemical characteristics of a controlled release composition, is reduced by complexing at least one type of metal cation with neurotoxin prior to contacting the neurotoxin with a polymer solution.

By the present invention, stabilized neurotoxin is stabilized against significant aggregation in vivo over the controlled release period. Significant aggregation is defined as an amount of aggregation resulting in aggregation of about 15% or more of the polymer encapsulated or polymer matrix incorporated neurotoxin. Preferably, aggregation is maintained below about 5% of the neurotoxin. More preferably, aggregation is maintained below about 2% of the neurotoxin present in the polymer.

In another embodiment, a neurotoxin controlled release composition also contains a second metal cation component, which is not contained in the stabilized neurotoxin particles, and which is dispersed within the polymer. The second metal cation component preferably contains the same species of metal cation, as is contained in the stabilized neurotoxin. Alternately, the second metal cation component can contain one or more different species of metal cation.

The second metal cation component acts to modulate the release of the neurotoxin from the polymeric matrix of the controlled release composition, such as by acting as a reservoir of metal cations to further lengthen the period of time over which the neurotoxin is stabilized by a metal cation to enhance the stability of neurotoxin in the composition.

A metal cation component used in modulating release typically contains at least one type of multivalent metal cation. Examples of second metal cation components suitable to modulate neurotoxin release, include, or contain, for instance, Mg(OH)₂, MgCO₃ (such as 4MgCO₃Mg(OH)₂5H₂O), ZnCO₃(such as 3Zn(OH)₂2ZnCO₃), CaCO₃, Zn₃ (C₆H₅O₇) 2, Mg(OAc)₂, MgSO₄, Zn(OAc)₂, ZnSO₄, ZnCl₂, MgCl₂ and Mg₃ (C₆H₅O₇)₂. A suitable ratio of second metal cation component-to-polymer is between about 1:99 to about 1:2 by weight. The optimum ratio depends upon the polymer and the second metal cation component utilized.

The neurotoxin controlled release composition of this invention can be formed into many shapes such as a film, a pellet, a cylinder, a disc or a microsphere. A microsphere, as defined herein, comprises a polymeric component having a diameter of less than about one millimeter and having stabilized neurotoxin dispersed therein. A microsphere can have a spherical, non-spherical or irregular shape. It is preferred that a microsphere be spherical in shape. Typically, the microsphere will be of a size suitable for injection. A preferred size range for microspheres is from about 1 to about 180 microns in diameter.

In the method of this invention for forming a composition for the controlled release of biologically active, non-aggregated neurotoxin, a suitable amount of particles of biologically active, stabilized neurotoxin are dispersed in a polymer solution.

A suitable polymer solvent, as defined herein, is solvent in which the polymer is soluble but in which the stabilized neurotoxin is are substantially insoluble and non-reactive. Examples of suitable polymer solvents include polar organic liquids, such as methylene chloride, chloroform, ethyl acetate and acetone.

To prepare biologically active, stabilized neurotoxin, neurotoxin is mixed in a suitable aqueous solvent with at least one suitable metal cation component under pH conditions suitable for forming a complex of metal cation and neurotoxin. Typically, the complexed neurotoxin will be in the form of a cloudy precipitate, which is suspended in the solvent. However, the complexed neurotoxin can also be in solution. In an even more preferred embodiment, neurotoxin is complexed with Zn⁺⁺.

Suitable pH conditions to form a complex of neurotoxin typically include pH values between about 5.0 and about 6.9. Suitable pH conditions are typically achieved through use of an aqueous buffer, such as sodium bicarbonate, as the solvent.

Suitable solvents are those in which the neurotoxin and the metal cation component are each at least slightly soluble, such as in an aqueous sodium bicarbonate buffer. For aqueous solvents, it is preferred that water used be either deionized water or water-for-injection (WFI).

The neurotoxin can be in a solid or a dissolved state, prior to being contacted with the metal cation component. Additionally, the metal cation component can be in a solid or a dissolved state, prior to being contacted with the neurotoxin. In a preferred embodiment, a buffered aqueous solution of neurotoxin is mixed with an aqueous solution of the metal cation component.

Typically, the complexed neurotoxin will be in the form of a cloudy precipitate, which is suspended in the solvent. However, the complexed neurotoxin can also be in solution. In a preferred embodiment, the neurotoxin is complexed with Zn⁺⁺.

The Zn⁺⁺ complexed neurotoxin can then be dried, such as by lyophilization, to form particulates of stabilized neurotoxin. The Zn⁺⁺ complexed neurotoxin, which is suspended or in solution, can be bulk lyophilized or can be divided into smaller volumes which are then lyophilized. In a preferred embodiment, the Zn⁺⁺ complexed neurotoxin suspension is micronized, such as by use of an ultrasonic nozzle, and then lyophilized to form stabilized neurotoxin particles. Acceptable means to lyophilize the Zn⁺⁺ complexed neurotoxin mixture include those known in the art.

In another embodiment, a second metal cation component, which is not contained in the stabilized neurotoxin particles, is also dispersed within the polymer solution.

It is understood that a second metal cation component and stabilized neurotoxin can be dispersed into a polymer solution sequentially, in reverse order, intermittently, separately or through concurrent additions. Alternately, a polymer, a second metal cation component and stabilized neurotoxin and can be mixed into a polymer solvent sequentially, in reverse order, intermittently, separately or through concurrent additions. In this method, the polymer solvent is then solidified to form a polymeric matrix containing a dispersion of stabilized neurotoxins.

A suitable method for forming an neurotoxin controlled release composition from a polymer solution is the solvent evaporation method is described in U.S. Pat. Nos. 3,737,337; 3,523,906; 3,691,090, and; 4,389,330. Solvent evaporation can be used as a method to form neurotoxin controlled release microparticles.

In the solvent evaporation method, a polymer solution containing a stabilized neurotoxin particle dispersion, is mixed in or agitated with a continuous phase, in which the polymer solvent is partially miscible, to form an emulsion. The continuous phase is usually an aqueous solvent. Emulsifiers are often included in the continuous phase to stabilize the emulsion. The polymer solvent is then evaporated over a period of several hours or more, thereby solidifying the polymer to form a polymeric matrix having a dispersion of stabilized neurotoxin particles contained therein.

A preferred method for forming neurotoxin controlled release microspheres from a polymer solution is described in U.S. Pat. No. 5,019,400. This method of microsphere formation, as compared to other methods, such as phase separation, additionally reduces the amount of neurotoxin required to produce a controlled release composition with a specific neurotoxin content.

In this method, the polymer solution, containing the stabilized neurotoxin dispersion, is processed to create droplets, wherein at least a significant portion of the droplets contain polymer solution and the stabilized neurotoxin. These droplets are then frozen by means suitable to form microspheres. Examples of means for processing the polymer solution dispersion to form droplets include directing the dispersion through an ultrasonic nozzle, pressure nozzle, Rayleigh jet, or by other known means for creating droplets from a solution.

The solvent in the frozen microdroplets is extracted as a solid and/or liquid into the non-solvent to form stabilized neurotoxin containing microspheres. Mixing ethanol with other non-solvents, such as hexane or pentane, can increase the rate of solvent extraction, above that achieved by ethanol alone, from certain polymers, such as poly(lactide-co-glycolide) polymers.

Yet another method of forming a neurotoxin implant, from a polymer solution, includes film casting, such as in a mold, to form a film or a shape. For instance, after putting the polymer solution containing a dispersion of stabilized neurotoxin into a mold, the polymer solvent is then removed by means known in the art, or the temperature of the polymer solution is reduced, until a film or shape, with a consistent dry weight, is obtained.

In the case of a biodegradable polymer implant, release of neurotoxin due to degradation of the polymer. The rate of degradation can be controlled by changing polymer properties that influence the rate of hydration of the polymer. These properties include, for instance, the ratio of different monomers, such as lactide and glycolide, comprising a polymer; the use of the L-isomer of a monomer instead of a racemic mixture; and the molecular weight of the polymer. These properties can affect hydrophilicity and crystallinity, which control the rate of hydration of the polymer. Hydrophilic excipients such as salts, carbohydrates and surfactants can also be incorporated to increase hydration and which can alter the rate of erosion of the polymer.

By altering the properties of a biodegradable polymer, the contributions of diffusion and/or polymer degradation to neurotoxin release can be controlled. For example, increasing the glycolide content of a poly(lactide-co-glycolide) polymer and decreasing the molecular weight of the polymer can enhance the hydrolysis of the polymer and thus, provides an increased neurotoxin release from polymer erosion. In addition, the rate of polymer hydrolysis is increased in non-neutral pH's. Therefore, an acidic or a basic excipient can be added to the polymer solution, used to form the microsphere, to alter the polymer erosion rate.

An implant within the scope of the present invention can be administered to a human, or other animal, by any non-systemic means of administration, such as by implantation (e.g. subcutaneously, intramuscularly, intracranially, intravaginally and intradermally), to provide the desired dosage of neurotoxin based on the known parameters for treatment with neurotoxin of various medical conditions, as previously set forth.

The specific dosage by implant appropriate for administration is readily determined by one of ordinary skill in the art according to the factor discussed above. The dosage can also depend upon the size of the tissue mass to be treated or denervated, and the commercial preparation of the toxin. Additionally, the estimates for appropriate dosages in humans can be extrapolated from determinations of the amounts of botulinum required for effective denervation of other tissues. Thus, the amount of botulinum A to be injected is proportional to the mass and level of activity of the tissue to be treated. Generally, between about 0.01 units per kilogram to about 35 units per kg of patient weight of a botulinum toxin, such as botulinum toxin type A, can be released by the present implant per unit time period (i.e. over a period of or once every 2-4 months) to effectively accomplish a desired intraocular therapeutic effect. Less than about 0.01 U/kg of a botulinum toxin does not have a significant therapeutic effect upon a muscle, while more than about 35 U/kg of a botulinum toxin approaches a toxic dose of a neurotoxin, such as a botulinum toxin type A. Careful preparation and placement of the implant prevents significant amounts of a botulinum toxin from appearing systemically. A more preferred dose range is from about 0.01 U/kg to about 25 U/kg of a botulinum toxin, such as that formulated as BOTOX®. The actual amount of U/kg of a botulinum toxin to be administered depends upon factors such as the extent (mass) and level of activity of the tissue to be treated and the administration route chosen. Botulinum toxin type A is a preferred botulinum toxin serotype for use in the methods of the present invention.

Preferably, a neurotoxin used to practice a method within the scope of the present invention is a botulinum toxin, such as one of the serotype A, B, C, D, E, F or G botulinum toxins. Preferably, the botulinum toxin used is botulinum toxin type A, because of its high potency in humans, ready availability, and known safe and efficacious use for the treatment of various disease and conditions.

The present invention includes within its scope the use of any Clostridial neurotoxin. For example, neurotoxins made by any of the species of the toxin producing Clostridium bacteria, such as Clostridium botulinum, Clostridium butyricum, and Clostridium beratti can be used or adapted for use in the methods of the present invention. Additionally, all of the botulinum serotypes A, B, C, D, E, F and G can be advantageously used in the practice of the present invention, although type A is the most preferred serotype, as explained above. Practice of the present invention can provide effective relief for from 1 day to about 5 or 6 years.

The present invention includes within its scope: (a) neurotoxin complex as well as pure neurotoxin obtained or processed by bacterial culturing, toxin extraction, concentration, preservation, freeze drying and/or reconstitution and; (b) modified or recombinant neurotoxin, that is neurotoxin that has had one or more amino acids or amino acid sequences deliberately deleted, modified or replaced by known chemical/biochemical amino acid modification procedures or by use of known host cell/recombinant vector recombinant technologies, as well as derivatives or fragments of neurotoxins so made, and includes neurotoxins with one or more attached targeting moieties for a cell surface receptor present on a cell.

Botulinum toxins for use according to the present invention can be stored in lyophilized or vacuum dried form in containers under vacuum pressure. Prior to lyophilization the botulinum toxin can be combined with pharmaceutically acceptable excipients, stabilizers and/or carriers, such as albumin. The lyophilized or vacuum dried material can be reconstituted with saline or water.

The present invention also includes within its scope the use of an implanted controlled release neurotoxin complex so as to provide therapeutic relief from an ocular disorder. Thus, the neurotoxin can be imbedded within, absorbed, or carried by a suitable polymer matrix which can be implanted or embedded subdermally so as to provide a year or more of delayed and controlled release of the neurotoxin to the desired target tissue. Implantable polymers which permit controlled release of polypeptide drugs are known, and can be used to prepare a botulinum toxin implant suitable for vitreal insertion. See e.g. Pain 1999;82(1):49-55; Biomaterials 1994;15(5):383-9; Brain Res 1990;515(1-2):309-11 and U.S. Pat. Nos. 6,022,554; 6,011,011; 6,007,843; 5,667,808, and; 5,980,945.

It is known that a significant water content of lyophilized tetanus toxoid can cause solid phase aggregation and inactivation of the toxoid once encapsulated within microspheres. Thus, with a 10% (grams of water per 100 grams of protein) tetanus toxoid water content about 25% of the toxin undergoes aggregation, while with a 5% water content only about 5% of the toxoid aggregates. See e.g. Pages 251, Schwendeman S. P. et al., Peptide, Protein, and Vaccine Delivery From Implantable Polymeric Systems, chapter 12 (pages 229-267) of Park K., Controlled Drug Delivery Challenges and Strategies, American Chemical Society (1997). Significantly, the manufacturing process for BOTOX® results in a freeze dried botulinum toxin type A complex which has a moisture content of less than about 3%, at which moisture level nominal solid phase aggregation can be expected.

A general procedure for making a pulsatile, biodegradable botulinum toxin implant is as follows. The implant can comprise from about 25% to about 100% of a polylactide which is a polymer of lactic acid alone. Increasing the amount of lactide in the implant can increases the period of time before which the implant begins to biodegrade, and hence increase the time to pulsatile release of the botulinum toxin from the implant. The implant can also be a copolymer of lactic acid and glycolic acid. The lactic acid can be either in racemic or in optically active form, and can be either soluble in benzene and having an inherent viscosity of from 0.093 (1 g. per 100 ml. in chloroform) to 0.5 (1 g. per 100 ml. in benzene), or insoluble in benzene and having an inherent viscosity of from 0.093 (1 g. per 100 ml in chloroform) to 4 (1 g. per 100 ml in chloroform or dioxin). The implant can also comprise from 0.001% to 50% of a botulinum toxin uniformly dispersed in carrier polymer.

Once implanted the implant begins to absorb water and exhibits two successive and generally distinct phases of neurotoxin release. In the first phase neurotoxin is released through by initial diffusion through aqueous neurotoxin regions which communicate with the exterior surface of the implant. The second phase occurs upon release of neurotoxin consequent to degradation of the biodegradable polymer (i.e. a polylactide). The diffusion phase and the degradation-induced phase are temporally distinct in time. When the implant is placed in an aqueous physiological environment, water diffuses into the polymeric matrix and is partitioned between neurotoxin and polylactide to form aqueous neurotoxin regions. The aqueous neurotoxin regions increase with increasing absorption of water, until the continuity of the aqueous neurotoxin regions reaches a sufficient level to communicate with the exterior surface of the implant. Thus, neurotoxin starts to be released from the implant by diffusion through aqueous polypeptide channels formed from the aqueous neurotoxin regions, while the second phase continues until substantially all of the remaining neurotoxin has been released.

Also within the scope of the present invention is an implant in the form of a suspension for use by intravitreal injection, prepared by suspending the neurotoxin encapsulated microspheres in a suitable liquid, such as physiological saline.

Applications

Examples of medical conditions of the eye which may be treated by the implants and methods of the invention include, but are not limited to, uveitis, macular edema, macular degeneration, retinal detachment, ocular tumors, fungal or viral infections, multifocal choroiditis, diabetic retinopathy, proliferative vitreoretinopathy (PVR), sympathetic opthalmia, Vogt Koyanagi-Harada (VKH) syndrome, histoplasmosis, uveal diffusion, and vascular occlusion. In one variation, the implants are particularly useful in treating such medical conditions as uveitis, macular edema, vascular occlusive conditions, proliferative vitreoretinopathy (PVR), and various other retinopathies.

Method of Implantation

The biodegradable implants may be inserted into the eye by a variety of methods, including placement by forceps, by trocar, or by other types of applicators, after making an incision in the sclera. In some instances, a trocar or applicator may be used without creating an incision. In a preferred variation, a hand held applicator is used to insert one or more biodegradable implants into the eye. The hand held applicator typically comprises an 18-30 GA stainless steel needle, a lever, an actuator, and a plunger.

The method of implantation generally first involves accessing the target area within the ocular region with the needle. Once within the target area, e.g., the vitreous cavity, the lever on the hand held device is depressed to cause the actuator to drive the plunger forward. As the plunger moves forward, it pushes the implant into the target area.

Extrusion Methods

The use of extrusion methods allows for large-scale manufacture of implants and results in implants with a homogeneous dispersion of the drug within the polymer matrix. When using extrusion methods, the polymers and botulinum toxins that are chosen are stable at temperatures required for manufacturing, usually not greater than about 40° C. Extrusion methods can use temperatures of about 25° C. to about 40° C.

Different extrusion methods may yield implants with different characteristics, including but not limited to the homogeneity of the dispersion of the botulinum toxin within the polymer matrix. For example, using a piston extruder, a single screw extruder, and a twin screw extruder will generally produce implants with progressively more homogeneous dispersion of the active. When using one extrusion method, extrusion parameters such as temperature, extrusion speed, die geometry, and die surface finish will have an effect on the release profile of the implants produced.

In one variation of producing implants by extrusion methods, the drug and polymer are first mixed at room temperature and then heated to a temperature range of about 25° C. to about 40° C., more usually to about 30° C. for a time period of about 0 to about 1 hour, more usually from about 0 to about 30 minutes, more usually still from about 5 minutes to about 15 minutes, and most usually for about 10 minutes. The implants are then extruded at a temperature of about 30° C.

In a preferred extrusion method, the powder blend of botulinum toxin and PLGA is added to a single or twin screw extruder preset at a temperature of about 25° C. to about 40° C., and directly extruded as a filament or rod with minimal residence time in the extruder. The extruded filament or rod is then cut into small implants having the loading dose of botulinum toxin appropriate to treat the medical condition of its intended use.

### EXAMPLES

The following examples illustrate embodiments and aspects of the present invention.

### Example 1

### Formation of Zinc⁺⁺ Stabilized Botulinum Neurotoxin

One hundred units of a neurotoxin, such as unreconstituted Botox®, is dissolved in sodium bicarbonate buffer (pH 6.0) to form a neurotoxin solution. A Zn⁺⁺ solution is prepared from deionized water and zinc acetate dihydrate and then added with gentle mixing to the neurotoxin solution to form a Zn⁺⁺ neurotoxin complex. The pH of the Zn⁺⁺ neurotoxin complex is then adjusted to between 6.5 and 6.9 by adding 1% acetic acid. A cloudy suspended precipitate, comprising insoluble Zn⁺⁺ stabilized neurotoxin is thereby formed. There is thereby prepared a botulinum toxin type A complex stabilized against significant aggregation upon subsequent incorporation into a polymeric implant matrix.

### Example 2

### Botulinum Neurotoxin Controlled Release Pellet

A botulinum neurotoxin suitable for incorporation into a polymer or polymerizable solution can be a botulinum toxin type A (such as Botox®), which is commercially available as a freeze dried or lyophilized powder. Additionally, various polymers and copolymers can be mixed and stored in a dry state with no effect on final implant performance. For example, an acrylate copolymer using an UV cured initiator. The botulinum neurotoxin can be complexed with Zn⁺⁺ as set forth in Example 1 above. The Zn⁺⁺ stabilized botulinum neurotoxin complex is then mixed with uncured acrylate copolymer, UV initiator and an acid (pH between 5.5 and 6.8). The mixture is placed into a glass or clear plastic pellet mold which allows penetration of UV light. The mold is placed into a temperature controlled water bath held at 2° C. The pellet is cured with UV light for approximately 50 seconds, packaged and sterilized. The duration and intensity of the UV curing are such that insignificant amount of neurotoxin are disrupted or denatured.

The size of the pellet and the concentration of the amount of botulinum neurotoxin within the pellet are defined by the desired application. When the pellet is implanted, the pellet is hydrated inside of the body, which can delay an initial burst of the botulinum neurotoxin from inside the implant. In this example the pellet effectiveness would be for approximately about 4 to about 6 months.

### Example 3

### Botulinum Neurotoxin Controlled Release Formulations

To increase the amount of time the pellet can effectively deliver a botulinum neurotoxin, multiple layers of materials can be used. Thus, the inner material can be made from a polyvinylpyrrolidone/methylmethacrylate copolymer. This material allows for sustaining a high concentration of neurotoxin complex. A suitable amount of neurotoxin is complexed with Zn⁺⁺ as set forth in Example 1 above and this complex is then mixed with uncured copolymer, low temperature initiator and an acid (pH between 5.5 and 6.8). The mixture is placed into a glass or plastic pellet mold. The mold is placed into a temperature controlled water bath at about 35 degrees C. for between about 6 hours and about 8 hours. This forms the reservoir of neurotoxin required for a prolonged, controlled release.

In order to prolong the release of the neurotoxin a second material can then cured around the initial pellet. This material is chosen for high molecular density and biocompatibility. Polymethylmethacrylate (PMMA) is an example of a material with this characteristic. The pellet (above) is placed into a mold (insertion molding) with uncured PMMA/low temperature initiator. A secondary coating of the uncured PMMA maybe necessary to assure uniform coating of the pellet. Preferably, the PMMA thickness is 0.5 mm. After forming, the outside of the pellet is coated with the desired initial burst concentration of neurotoxin. The PMMA layer will be sufficiently thick to allow for a delay (up to 3 months) of the neurotoxin in the reservoir. When the neurotoxin reaches the surface of the implant a second large burst of neurotoxin is obtained. This secondary burst will then be followed by a slowly decreasing release rate of the neurotoxin for approximately 3 months. In this example the pellet effectiveness is for about 7 to about 9 months.

### Example 4

### Method for Making a Biodegradable Botulinum Toxin Implant

A biodegradable implant comprising botulinum toxin and a suitable carrier polymer can be prepared by dispersing an appropriate amount of a stabilized botulinum toxin preparation (i.e. non-reconstituted BOTOX®) into a continuous phase consisting of a biodegradable polymer in a volatile organic solvent, such as dichloromethane. Both PLGA and polyanhydrides are insoluble in water and require use of organic solvents in the microencapsulation process.

The polymer is dissolved in an organic solvent such as methylene chloride or ethyl acetate to facilitate microsphere fabrication. The botulinum toxin is then mixed by homogenization or sonication to form a fine dispersion of toxin in polymer/organic solvent, as an emulsion when an aqueous protein solution is used or as a suspension when a solid protein formulation is mixed with the polymer-organic solvent solution. The conventional processes for microsphere formation are solvent evaporation and solvent (coacervation) methods. Microspheres can be formed by mixing the preformed suspension of protein drug with polymer-organic solvent, with water containing an emulsifier (i.e. polyvinyl alcohol). Additional water is then added to facilitate removal of the organic solvent from the microspheres allowing them to harden. The final microspheres are dried to produce a free flowing powder.

The polymer used can be PLA, PGA or a co-polymer thereof. Alternately, a botulinum toxin incorporating polymer can be prepared by emulsifying an aqueous solution of the neurotoxin (i.e. reconstituted BOTOX®) into the polymer-organic phase (obtaining thereby a W/O emulsion). With either process a high speed stirrer or ultrasound is used to ensure uniform toxin mixing with the polymer. Microparticles 1-50 μm in diameter can be formed by atomizing the emulsion into a stream of hot air, inducing the particle formation through evaporation of the solvent (spray-drying technique). Alternately, particle formation can be achieved by coacervation of the polymer through non-solvent addition, e.g. silicon oil (phase separation technique) or by preparing a W/O/W emulsion (double emulsion technique).

The pH of the casting or other solution in which the botulinum toxin is to be mixed is maintained at pH 4.2-6.8, because at pH above about pH 7 the stabilizing nontoxin proteins can dissociate from the botulinum toxin resulting in gradual loss of toxicity. Preferably, the pH is between about 5-6. Furthermore the temperature of the mixture/solution should not exceed about 35 degrees Celsius, because the toxin can be readily detoxified when in a solution/mixture heated above about 40 degrees Celsius.

Methods for freezing droplets to form microparticles include directing the droplets into or near a liquefied gas, such as liquid argon and liquid nitrogen to form frozen microdroplets which are then separated from the liquid gas. The frozen microdroplets can then be exposed to a liquid non-solvent, such as ethanol, or ethanol mixed with hexane or pentane.

A wide range of sizes of botulinum toxin implant microparticles can be made by varying the droplet size, for example, by changing the ultrasonic nozzle diameter. If very large microparticles are desired, the microparticles can be extruded through a syringe directly into the cold liquid. Increasing the viscosity of the polymer solution can also increase microparticle size. The size of the microparticles can be produced by this process, for example microparticles ranging from greater than about 1000 to about 1 micrometers in diameter.

### Example 5

### Method for Making a Polyanhydride Botulinum Toxin Implant

A biodegradable polyanhydride polymer can be made as a copolymer of poly-carboxyphenoxypropane and sebacic acid in a ratio of 20:80. Polymer and a botulinum toxin (such as non-reconstituted BOTOX®) can be co-dissolved in methylene chloride at room temperature and spray-dried into microspheres, using the technique of Example 1. Any remaining methylene chloride can be evaporated in a vacuum desiccator.

Depending upon the implant size desired and hence the amount of botulinum toxin, a suitable amount of the microspheres can be compressed at about 8000 p.s.i. for 5 seconds or at 3000 p.s.i. for 17 seconds in a mold to form implant discs encapsulating the neurotoxin. Thus, the microspheres can be compression molded pressed into discs 1.4 cm in diameter and 1.0 mm thick, packaged in aluminum foil pouches under nitrogen atmosphere and sterilized by 2.2×10⁴ Gy gamma irradiation. The polymer permits release of the botulinum toxin over a prolonged period, and it can take more than a year for the polymer to be largely degraded.

### Example 6

### Water in Oil Method for Making a Biodegradable Botulinum Toxin Implant

A pulsatile release botulinum toxin implant can be made by dissolving a 80:20 copolymers of polyglycolic acid and the polylactic acid can in 10% w/v of dichloromethane at room temperature with gentle agitation. A water-in-oil type emulsion can then be made by adding 88 parts of the polymer solution to 1 part of a 1:5 mixture of Tween 80 (polyoxyethylene 20 sorbitan monooleate, available from Acros Organics N.V., Fairlawn, N.J.) and Span 85 (sorbitan trioleate) and 11 parts of an aqueous mixture of 75 units of BOTOX® (botulinum toxin type A complex) and Quil A (adjuvant). The mixture is agitated using a high-speed blender and then immediately spray-dried using a Drytec Compact Laboratory Spray Dryer equipped with a 60/100/120 nozzle at an atomizing pressure of 15 psi and an inlet temperature of 65 degrees C. The resultant microspheres have a diameter of about 20 μm diameter and are collected as a free-flowing powder. Traces of remaining organic solvent are removed by vacuum evaporation.

### Example 7

### Reduced Temperature Method for a Biodegradable Pulsatile Botulinum Toxin Implant

A pulsatile release botulinum toxin delivery system can be made at a low temperature so as to inhibit toxin denaturation as follows. 0.3 g of PLGA/ml of methylene chloride or ethyl acetate is mixed with 0.1 ml of neurotoxin solution/ml of the polymer-organic solution at a reduced temperature (2-8 degrees C.). A first set of botulinum toxin incorporating microspheres made, as set forth in Example 1 (the polymer solution is formed by dissolving the polymer in methylene chloride), from a 75:25 lactide:glycolide polymer with an inherent viscosity (dL/g) of about 0.62 (available form MTI) can degrade in vivo, and hence exhibit a pulsed release of the botulinum toxin, at about ninety days post implantation and extending over 2-4 weeks. A second set of, botulinum toxin incorporating microspheres made, as previously set forth (the polymer solution is formed by dissolving the polymer in ethyl acetate), from a 100:0 lactide:glycolide polymer with an inherent viscosity of about 0.22 (available form MTI) can degrade in vivo, and hence exhibit a burst release of the botulinum toxin, at about one hundred and eighty days post implantation. A third set of, botulinum toxin incorporating microspheres made, as previously set forth (the polymer solution is formed by dissolving the polymer in methylene chloride, from a 95:5 poly(DL-lactide):glycolide polymer, can degrade in vivo, and hence exhibit a burst release of the botulinum toxin, at about two hindered and seventy days post implantation. A fourth set of botulinum toxin incorporating microspheres made, as previously set forth (the polymer solution is formed by dissolving the polymer in methylene chloride), from a 100:0 poly(DL-lactide):glycolide polymer can degrade in vivo, and hence exhibit a burst release of the botulinum toxin, at about twelve months post implantation. Polymers can be obtained from Medisorb Technologies International (MTI).

A suspension or compression molded pellet which combines the four specified sets of botulinum toxin encapsulated microspheres can exhibit pulsatile release the neurotoxin. Local administration of botulinum toxin at the time of implantation (i.e. day zero) is provided by the initial burst release from the implanted microspheres.

### Example 8

### Method for Inserting an Implant into the Vitreous

An implant can be surgically implanted into the posterior segment of a human eye through an incision in the pars plana inferotemporally. A sterile trocar, preloaded with a 5 unit or 10 unit botulinum toxin ocular implant can be inserted 5 mm through the sclerotomy, and then retracted with the push wire in place, leaving the implant in the posterior segment. Sclerae and conjunctivae can then be closed using a 7-0 Vicryl suture. After closure, the suture knot can be buried and subconjunctival and topical antibiotics used prophylactically. Such an intraviteal implant can be used to treat a variety of ocular disorders such as macular edema, uveitis, macular degeneration, retinal detachment, ocular tumors, ocular fungal or viral infections, multifocal choroiditis, diabetic retinopathy, proliferative vitreoretinopathy, sympathetic opthalmia, Vogt Koyanagi-Harada syndrome, histoplasmosis, uveal diffusion, and ocular vascular occlusion.

### Example 9

### In Vivo Release of Botulinum Toxin Type A from a 5 Unit Botulinum Toxin Compressed Tablet Implants

An high initial release but generally lower intravitreal concentration of botulinum toxin from compressed tablet implants can be demonstrated as compared to extruded implants. The volume of the vitreous of the posterior chamber of the rabbit eye is about 1.5 ml and for the human eye about 3.9 ml. A 5 unit compressed tablet implant can be placed in the right eye of New Zealand White Rabbits as described in Example 8. Vitreous samples can be taken periodically and assayed by LC/MS/MS to determine in vivo botulinum toxin delivery performance. The botulinum toxin can reach detectable mean intravitreal concentrations from day 1 (i.e. 1 unit/ml vitreous) through day 35 (i.e. 0.02 units/ml vitreous), and the intravitreal concentration of botulinum toxin can gradually decrease over time.

In addition to the vitreous samples, aqueous humor and plasma samples can also be taken. The 5 unit tablet can show a gradual decrease in aqueous humor botulinum toxin concentrations over time, with the levels of botulinum toxin in the aqueous humor being strongly correlated with the levels of botulinum toxin in the vitreous humor, but at a much lower level (approximately 10-fold lower). Only trace amounts of botulinum toxin can be found in the plasma.

### Example 10

### In Vivo Release of Botulinum Neurotoxin Type A from 5 Unit Botulinum Toxin Extruded Implants

Lower initial release and generally more sustained intravitreal concentration of botulinum toxin can occur from extruded implants. A 5 unit extruded implant can be placed in the right eye of New Zealand White Rabbits as described in Example 8. Vitreous samples can be taken periodically and assayed by LC/MS/MS to determine in vivo botulinum toxin delivery performance. The 5 unit extruded implant can show detectable mean vitreous humor concentrations on day 1 through day 28. In addition to the vitreous samples, aqueous humor and plasma samples can also be taken to show detectable mean botulinum toxin aqueous humor concentrations at day 1 through day 42. On the whole, the levels of botulinum toxin in the aqueous can strongly correlate with the levels of botulinum toxin in the vitreous humor, but at a much lower level (approximately 10-fold lower). Only trace amounts of botulinum toxin may be found in the plasma.

### Example 11

### In Vivo Release of Botulinum Toxin Type A from 10 Unit Botulinum Toxin Compressed Tablet Implants

A the high initial release but generally lower intravitreal concentration of botulinum toxin from compressed tablet implants can be demonstrated as compared to extruded implants. A 10 unit compressed tablet implant can be placed in the right eye of New Zealand White Rabbits as described in Example 8. Vitreous samples can be taken periodically and assayed by LC/MS/MS to determine in vivo botulinum toxin delivery performance. The botulinum toxin can reach detectable mean intravitreal concentrations from day 1 (2 unit/ml vitreous) through day 35 (0.04 units/ml vitreous), and the intravitreal concentration of botulinum toxin gradually decreased over time.

In addition to the vitreous samples, aqueous humor and plasma samples can also be taken. The 10 unit tablet can show a gradual decrease in aqueous humor botulinum toxin concentrations over time, with the levels of botulinum toxin in the aqueous humor being strongly correlated with the levels of botulinum toxin in the vitreous humor, but at a much lower level (approximately 10-fold lower). Only trace amounts of botulinum toxin can be found in the plasma.

### Example 12

### In Vivo Release of Botulinum Neurotoxin Type A from 10 Unit Botulinum Toxin Extruded Implants

Lower initial release and generally more sustained intravitreal concentration of botulinum toxin can occur from extruded implants. A 10 unit extruded implant can be placed in the right eye of New Zealand White Rabbits as described in Example 8. Vitreous samples can be taken periodically and assayed by LC/MS/MS to determine in vivo botulinum toxin delivery performance. The 10 unit extruded implant can show detectable mean vitreous humor concentrations on day 1 through day 28. In addition to the vitreous samples, aqueous humor and plasma samples can also be taken to show detectable mean botulinum toxin aqueous humor concentrations at day 1 through day 42. On the whole, the levels of botulinum toxin in the aqueous can strongly correlate with the levels of botulinum toxin in the vitreous humor, but at a much lower level (approximately 10-fold lower). Only trace amounts of botulinum toxin may be found in the plasma.

An advantage of the present controlled release formulations for botulinum neurotoxins include long term, consistent therapeutic levels of neurotoxin at the target tissue. The advantages also include increased patient compliance and acceptance by reducing the required number of ocular injections.

All references, articles, publications and patents and patent applications cited herein are incorporated by reference in their entireties.

Although the present invention has been described in detail with regard to certain preferred methods, other embodiments, versions, and modifications within the scope of the present invention are possible. For example, a wide variety of neurotoxins can be effectively used in the methods of the present invention. Additionally, the present invention includes intravitreal administration methods wherein two or more neurotoxins, such as two or more botulinum toxins, are administered concurrently or consecutively via an ocular implant. For example, botulinum toxin type A can be administered via implant until a loss of clinical response or neutralizing antibodies develop, followed by administration via implant of a botulinum toxin type B or E. Alternately, a combination of any two or more of the botulinum serotypes A-G can be locally administered to control the onset and duration of the desired therapeutic result. Furthermore, non-neurotoxin compounds can be administered prior to, concurrently with or subsequent to administration of the neurotoxin via implant so as to provide an adjunct effect such as enhanced or a more rapid onset of denervation before the neurotoxin, such as a botulinum toxin, begins to exert its therapeutic effect.

The present invention also includes within its scope the use of a neurotoxin, such as a botulinum toxin, in the preparation of a medicament, such as a controlled release intraviteal implant, for the treatment of a an ocular disorder.

Accordingly, the spirit and scope of the following claims should not be limited to the descriptions of the preferred embodiments set forth above.

I claim:

1. An ocular implant for treating a medical condition of an eye, the ocular implant comprising:
(a) a carrier, and;
(b) a botulinum neurotoxin associated with the carrier, thereby forming an ocular implant, wherein a therapeutic amount of the botulinum neurotoxin can be released from the carrier upon implantation of the ocular implant into an eye of a patient to thereby treat a medical condition of an eye.

2. The ocular implant of claim 1, wherein the implant releases no more than about 15 percent of the botulinum neurotoxin from the carrier during the first twenty four hours after implantation of the ocular implant into an eye of a patient.

3. The ocular implant of claim 1, wherein the implant releases more than about 80 percent of the botulinum neurotoxin from the carrier within the first twenty eight days after implantation of the implant into an eye of a patient.

4. The ocular implant of claim 1, wherein the carrier is substantially biodegradable.

5. The ocular implant of claim 1 wherein the botulinum neurotoxin is selected from the group consisting of botulinum neurotoxin serotypes A, B, C, D, E, F and G.

6. A biodegradable ocular implant for treating a medical condition of the eye, the biodegradable ocular implant comprising:
(a) a biodegradable carrier, and;
(b) a botulinum neurotoxin associated with the biodegradable carrier, thereby forming a biodegradable ocular implant, wherein the implant releases no more than about 15 percent of the botulinum neurotoxin from the carrier during the first twenty four hours after implantation of the biodegradable implant into an eye of a patient and the implant releases more than about 80 percent of the botulinum neurotoxin from the carrier within the first twenty eight days after implantation of the implant into an eye of a patient.

7. The biodegradable implant of claim 6 wherein the botulinum toxin comprises from about 10 to about 90 percent by weight of the implant.

8. A method for treating an ocular disease, the method comprising the step of implanting into an eye of a patient a biodegradable implant comprising a botulinum neurotoxin associated with a carrier.

9. The method of claim 8, wherein the ocular disease is selected from the group consisting of uveitis, macular edema, macular degeneration, retinal detachment, ocular tumors, ocular fungal infections, ocular viral infections, multifocal choroiditis, diabetic retinopathy, proliferative vitreoretinopathy, sympathetic opthalmia, Vogt Koyanagi-Harada syndrome, histoplasmosis, uveal diffusion, and vascular occlusion.

10. The method of claim 8 wherein the biodegradable implant is implanted into a location in the eye selected from the group consisting of the anterior chamber, the posterior chamber, the vitreous cavity, the choroid, the suprachoroidal space, the conjunctiva, the subconjunctival space, the episcleral space, the intracorneal space, the epicorneal space, the sclera, the pars plana, surgically-induced avascular regions, the macula, and the retina.

11. The method of claim 10 wherein the location is the vitreous cavity.

12. The method of claim 11 wherein the step of implanting the biodegradable implant results in an approximately 10-fold less concentration of the botulinum toxin in the aqueous humor of the eye into which the implant was implanted as compared to the concentration of the botulinum toxin in the vitreous humor of the eye into which the implant was implanted.

13. A method for treating an ocular disease, the method comprising the step of implanting into the vitreous cavity of an eye of a patient a biodegradable implant comprising a botulinum neurotoxin associated with a carrier, wherein the step of implanting the biodegradable implant results in an approximately 10-fold less concentration of the botulinum toxin in the aqueous humor of the eye into which the implant was implanted as compared to the concentration of the botulinum toxin in the vitreous humor of the eye into which the implant was implanted.
